(19) United States
(12) Patent Application Publication
(10) Pub. No.: US 2006/0253126 A1
(43) Pub. Date: Nov. 9, 2006

(54) Endoluminal suturing device and method

(75) Inventors: David B. Bjerken, Marietta, GA (US); Vincente J. Fuillerat, Lakeland, FL (US)

Correspondence Address: MCKENNA LONG & ALDRIDGE LLP, 1900 K STREET, NW, WASHINGTON, DC, 20006, US

(73) Assignee: Bernard Medical, LLC, Marietta, GA (US)

(21) Appl. No.: 11/267,266

(22) Filed: Nov. 7, 2005

Related U.S. Application Data
(60) Provisional application No. 60/677,355, filed on May 4, 2005.
(60) Provisional application No. 60/677,345, filed on May 4, 2005.
(60) Provisional application No. 60/697,544, filed on Jul. 11, 2005.
(60) Provisional application No. 60/698,941, filed on Jul. 14, 2005.

(51) Int. Cl. A61B 17/10

(52) U.S. Cl. 606/139

(57) ABSTRACT

A suturing device for suturing within a subject is provided. The suturing device includes an enclosure defining at least one suction port, wherein the suction port receives tissue within the enclosure to be sutured; at least one cannula arranged at least partially within the enclosure; and at least one needle arranged within the at least one cannula, wherein the needles are adapted to be pushed through the cannula and directed through the tissue within the enclosure to provide a suture to the tissue.

### CROSS-REFERENCE TO RELATED APPLICATIONS

This application claims priority to Provisional Patent Application No. 60,677,345, filed May 4, 2005; Provisional Patent Application No. 60/677,355, filed May 11, 2005; Provisional Patent Application No. 60/697,544, filed Jul. 11, 2005; and Provisional Patent Application No. 60/698,941, filed Jul. 14, 2005. The disclosures of each of the above-mentioned applications are hereby incorporated by reference.

### BACKGROUND OF THE INVENTION

1. Field of the Invention

The present invention relates generally to suturing devices and methods for using the suturing device to place suture or tissue fastening material within tissue, for example, within a body organ.

2. Discussion of the Related Art

Various types of surgical procedures are currently performed to investigate, diagnose, and treat diseases and conditions within patients, including conditions and diseases within the gastrointestinal system and within the heart and great vessels within the thorax. Procedures include, for example, the placement of sutures involved with the treatment of many kinds of conditions and diseases. A suture is any fastening material. Conventionally the placement of sutures involves an invasive surgery to access the suture areas. Such procedures are time consuming, involve difficult placements of suture, and subject the patient to trauma and prolonged recovery.

U.S. Pat. Nos. 6,464,707 and 6,558,400 are hereby incorporated by reference in their entirety.

### SUMMARY OF THE INVENTION

The present invention includes a suturing device for suturing within a subject, and includes an enclosure defining at least one suction port for receiving tissue within the enclosure to be sutured, at least one cannula arranged at least partially within the enclosure, and at least one needle arranged within the at least one cannula. The needles are adapted to be pushed through the cannula and directed through the tissue within the enclosure to provide a suture to the tissue.

### BRIEF DESCRIPTION OF THE DRAWINGS

FIG. 1 illustrates a view of an exemplary embodiment of the present invention.

FIG. 2 illustrates one example of a tube used in accordance with the present invention.

FIG. 3 illustrates another example of a tube used in accordance with the present invention.

FIG. 4 illustrates another example of a tube used in accordance with the present invention.

FIG. 5 illustrates another example of a tube used in accordance with the present invention.

FIG. 6 illustrates another example of a tube used in accordance with the present invention.

FIG. 7 illustrates another example of a tube used in accordance with the present invention.

FIGS. 8A and 8B illustrate another example of a tube used in accordance with the present invention.

FIGS. 9A and 9B illustrate another example of a tube used in accordance with the present invention.

FIGS. 10A and 10B illustrate another example of a tube used in accordance with the present invention.

FIGS. 11A, 11B, and 11C illustrate another example of a tube used in accordance with the present invention.

FIGS. 12A and 12B illustrate an example of an expandable tube used in accordance with the present invention.

FIG. 13 illustrate an embodiment of the present invention with two cannulas.

FIG. 14 illustrate an embodiment of the present invention with a single cannula.

FIG. 15 illustrate an embodiment of the present invention with a single cannula utilized with a needle push-rod in the cannula.

FIGS. 16A and 16B illustrate an embodiment of the present invention with an extendable cannula.

FIG. 17A illustrates an embodiment of the present invention with an endoscope.

FIG. 17B illustrates an embodiment of the present invention suitable for placing sutures in any direction.

FIGS. 18A and 18B illustrate an embodiment of the present invention with an expandable device.

FIGS. 19A, 19B, and 19C illustrate another embodiment of the present invention with an expandable device.

FIG. 20 is an example of a needle and suture used in accordance with the present invention.

FIG. 21 is another example of a needle and suture used in accordance with the present invention.

FIGS. 22A-C is an example of a needle, suture and cannula configuration used in accordance with the present invention.

FIGS. 23A-D is another example of a needle and suture configuration used in accordance with the present invention.

FIGS. 24A-D is another example of a needle and suture configuration used in accordance with the present invention.

FIG. 25A-C illustrate an exemplary embodiment of the present invention.

FIG. 26A-C illustrate another exemplary embodiment of the present invention.

FIG. 27A-E illustrate a view of an exemplary embodiment of the present invention that utilizes push-rods.

FIG. 28A-F illustrate a view of another exemplary embodiment of the present invention that utilizes rollers.

FIGS. 29A-C illustrate an exemplary embodiment of the present invention that utilizes push-rods to place horizontal sutures.

FIGS. 30A and 30B illustrate an example of a needle and suture used in the embodiment shown in FIG. 29.

FIG. 31 illustrates another exemplary embodiment of the present invention.

FIGS. 32A-C illustrate an example of a method of practicing the present invention.

FIG. 33 illustrates another example of a method of practicing the present invention.

FIGS. 34A-D illustrate an example of another method of practicing the present invention.

FIGS. 35A-B illustrate an example of another method of practicing the present invention.

FIGS. 36A and 36B illustrate an example of another method of practicing the present invention

FIGS. 37A and 37B illustrate an example of another method of practicing the present invention

FIGS. 38A38C illustrate an example of another method of practicing the present invention.

FIGS. 39A and 39B illustrate an example of another method of practicing the present invention.

FIGS. 40A-K illustrate an example of another method of practicing the present invention.

FIGS. 41A-H illustrate an example of another method of practicing the present invention.

FIGS. 42A and 42B illustrate an example of another method of practicing the present invention.

FIGS. 43A-D illustrate an example of another method of practicing the present invention.

FIG. 44 illustrate another embodiment of the present invention.

FIGS. 45A-D illustrate another example of an embodiment of the present invention in which the needle includes a barb

FIGS. 46A-D illustrate another example of an embodiment of the present invention in which the needle includes a barb

FIGS. 47A-B illustrate another example of an embodiment of the present invention in which the needle includes a barb.

### DETAILED DESCRIPTION OF THE INVENTION

The present invention is more particularly described in the following examples with reference to the accompanying drawings that are intended as illustrative only since numerous modifications and variations therein will be apparent to those skilled in the art.

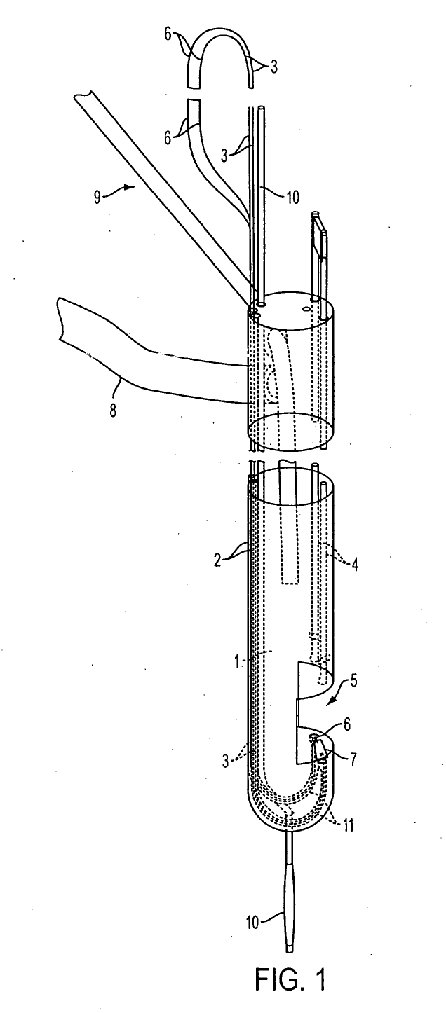

As shown in the exemplary embodiment of FIG. 1, the suturing device of the present invention may include an outer tube 1. The tube 1 has a flexibility to help facilitate insertion, internal navigation and positioning. The tube 1 can be transparent, translucent, or opaque. The tube 1 has a diameter and flexibility that is amenable for insertion into a natural body orifice, such as the mouth or anus, or into a surgical incision or existing stoma. The diameter can be, for example, about 5 mm to about 22 mm for oral insertion and about 5 mm to about 35 mm for anal insertion. For cardiac and vascular applications, the diameter can range from about 3 mm to about 35 mm in diameter.

In this embodiment, the tube 1 has a length is sufficient to span the distance from its place of insertion to the targeted surgical location. For example, in gastrointestinal (GI) uses, the tube 1 can be approximately 2 to 3 feet. This length enables the device to reach several organs within the GI tract or within the abdominal cavity while a proximal end of the tube remains outside the patient's body and accessible by the operator.

The tube 1 can be circular in its cross-section or it may have a non-circular cross-section. Possible cross-sectional shapes are oval, rectangular, or irregular, such as the shape of a mitral valve annulus.

As shown in FIG. 1, the tube I can house an endoscope 9. The device may include and be connected to the endoscope 9 or the endoscope 9 may work in concert with the device.

Generally, an operator manipulates the tube 1 to place the device at the desired location. The endoscope contained within the flexible tube of the device may direct the device to its target by applying force to the wall of the tube, thereby steering the device in the direction in which the force is applied. Alternatively, the tube 1 may have the ability to direct or steer itself by using various methods of steering. For example, a balloon catheter 10 can run parallel within or along a side of the tube. The catheter 10 may be endoscopically placed in a defect, annulus, valve, or outlet, and inflated to hold the device in place. The tube 1 can then be slid down the catheter 10 to be positioned and maintained in the desired location. In vascular applications, the device can be directed in a similar fashion following a guide wire. The tube 1 can incorporate radio opaque markers to enable visualization using fluoroscopy. In yet another embodiment, wires or cables can be used by varying tensions to turn the device within the closed organ or space.

The tube 1 is in fluid communication with a vacuum source 8 and can include one or more suction ports 5. The port 5 is generally located near the distal end of the tube, although other locations are possible. The suction port 5 is designed to draw tissue into a bore of the tube 1 when a vacuum is applied to the tube 1. Optionally, the device can include a sleeve inside or outside of the tube to control the size of the suction port 5.

One or more cannulas are arranged within the tube 1. Two sets of cannulas 2,4 are shown in FIG. 1. In this embodiment, a set of cannula includes a delivery cannula 2 and a corresponding receiving cannula 4 arranged on opposite sides of the suction port 5. Generally, a cannula is a tubular passageway though which material can travel in either a forward or backward direction. The cannulas 2,4 have an internal diameter adequate to contain one or more needles and accompanying sutures. The cannulas 2,4 are utilized to direct the needles to an intended point of incorporation with tissue. The cannulas 2,4 also direct the force necessary to maintain the needle in a forward or backward direction and contain or prohibit lateral movement and bending of the needle. The cannulas 2,4 assist in guiding the needles.

The cannulas 2,4 can be formed, molded, and/or cast as part of the tube 1, or can be independent components inserted into the tube. The cannulas 2,4 can be flexible, but may optionally have rigid sections as necessary to allow turning and targeting of the needles.

The cannulas 2,4 can be configured within the tube 1 in many ways. In the embodiment shown in FIG. 1, needles 3 can be pushed by an operator through the delivery cannulas 2, down the length of the tube, then directed upward at the bend 11 at the distal end of the tube 1. The needles 3 then pass out of the delivery cannulas 2 and traverse the suction port 5 such that the needles 3 penetrate the tissue drawn into the bore by the vacuum 8. The cannulas may have flaired or trumpet shaped openings to assist in receiving the needle once it has traversed the suction opening. The needles 3 completely traverse the tissue within the suction port 5, and may continue up the bore of the tube 1 or enter the receiving cannulas 4 at or near the upper side of the suction port 5. This creates a “bite” or suture of tissue.

If two or more suture bites are required, such as when the desired result is to attach one area of tissue to a second, the cannulas 2, 4 can be reloaded, as discussed in further detail below. The cannulas 2,4 can also be loaded with suture needles 3 in several different configurations, as descried in further detail below.

The tube 1 can have a constant diameter, or in alternative embodiments, the tube can expand and contract to aid in insertion and withdrawal. The ability to expand and contract is useful for large organs, such as the stomach, or for insertion areas that require a small diameter.

The suturing device of the present invention generally utilizes long suture needles made of a material that has the properties of shape memory, such as Nitinol. Nitinol is a nickel and titanium alloy that quickly returns to an original configuration after being flexed. Other materials can also be used, such as stainless steel. The suture needles can be of adequate length to reach a suturing site, via a natural body orifice such as the mouth or anus, or an incision or stoma, and return back out of the device. As such, the needles are typically at least twice the length of the tube of the device. As an example, a needle utilized for GI applications can be approximately 6 feet long. The needles can be attached to suture material to deliver and incorporate suture material into tissue that the needle traverses.

As shown in FIG. 4A, in one embodiment of the present invention, the needle is straight. The needle has a flexibility to follow the path within a cannula, which may include various turns and loops without losing its original shape. The straight shape of the needle allows it to exit the cannula, transect a port or ports in the tube such as a suction port, and proceed in the direction in which it has been directed.

The needle can be longer or shorter depending on the desired application, such as cardiac, vascular, gynecological, proctological, pulmonary, and general surgical procedures. The needle may have a distal tip or end that is made of a material that is more rigid, such as steel or titanium. The needles may have differing diameter or gauge depending upon the application. By way of example, vascular anastomosis generally requires relatively thin needles, for example, needles with a diameter of about 0.1 mm to about 0.5 mm. The needle may also have an original configuration other than straight, such as having a bend, curve or coil. The elongated needle may also have a detachable tip, with the detachable tip being attached to suture and the elongated shaft or wire serving as a pushrod.

In some embodiments, the device does not use a vacuum source. The tube may be of a size such that the tissue envelops or enters into a port without the need of a vacuum. This embodiment can be useful, for example, in a closure application.

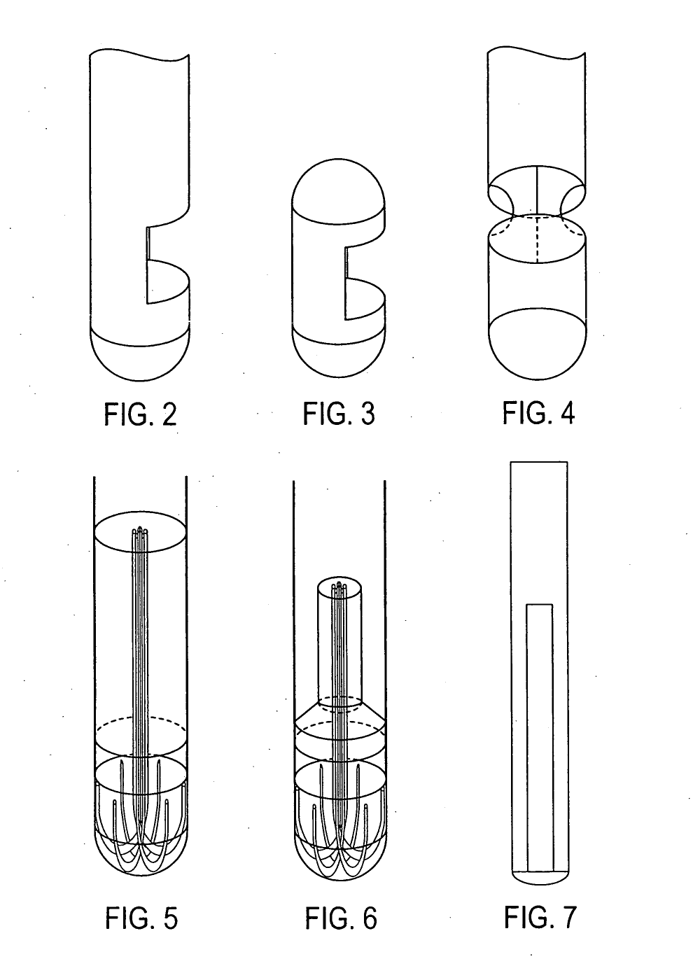

FIGS. 2-12B illustrate examples of different port and tube configurations.

A partial circumferential port is illustrated in FIG. 2. To form a partial circumferential suction port, a part of the tube can be removed to form the port. The width of the partial circumferential port can be varying sizes, for example, less than 1° to almost 360° of the circumference of the tube, or preferably, 90° to 180° of the circumference. The longitudinal length can vary depending upon the application. Generally the length will be from 5 mm to 25 mm.

In the embodiment of FIG. 3, the tube is capsule shaped with enclosed ends.

As shown in FIG. 5, a circumferential suction port may be used for putting sutures around an annulus or the circumference of a lumen. This port may actually be formed by two tubular segments held together by one or more vertical struts. The struts hold the two tubular segments at a predetermined distance. In another embodiment, the tubular segments are held together and at a determined distance apart from each other by an internal member or members running down the center of the enclosure.

In alternate embodiments, such as that shown in FIG. 4, the struts may have an inward curvature. The curvature allows tissue to be drawn into the bore of the tube about the entire circumference.

The device shown in FIG. 5 can be used for closing defects or incisions, or for reducing the size of an annulus or outlet. In this embodiment, the circumferential suction port uses removable struts. The struts can run on opposing sides of a tube's circumferential port. The distal and proximal tube segments can be held together by an internal member or members, such as the delivery cannulas running down the center of the tube. The removable struts define the axis of the closing suture line. Once the suction has been applied and the sutures have been deployed and are incorporated into the orifice, defect, or outlet, the struts may be raised and lifted out of the way. This prevents the sutures from being entrapped within the suction port of the device and will allow for the easy removal of the device from the site. The removable struts may be formed by flexible shafts or wires running in cannulas or channels designed to hold the shafts or wires in place until they needed to be removed.

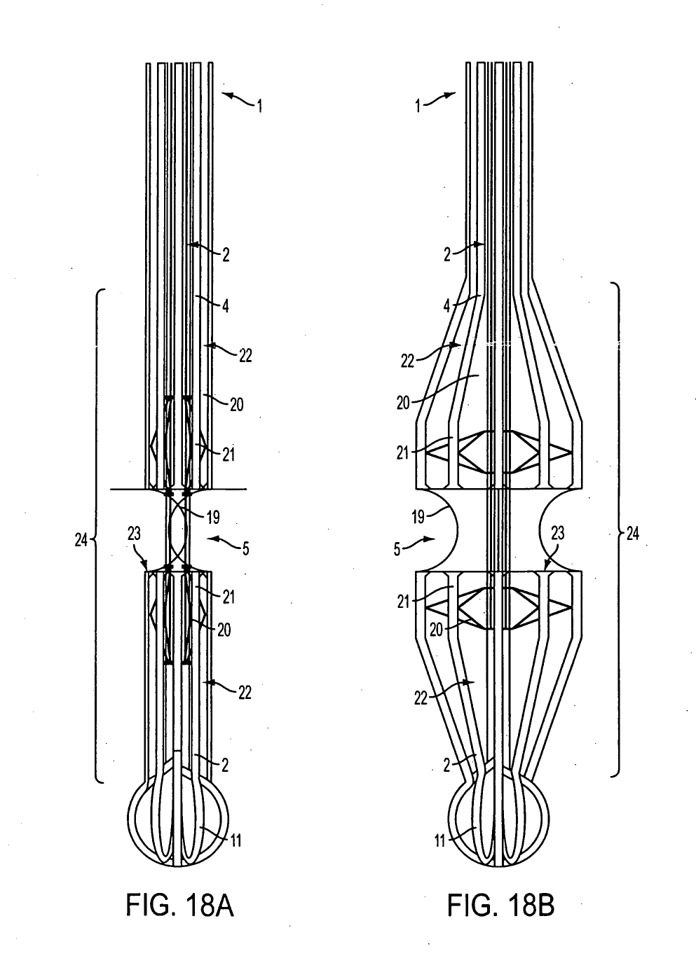

FIG. 6 and FIGS. 18A-B show an embodiment that includes a circumferential suction opening for a circumferential suture placement. This embodiment enables anastomosis, connection of tissue to tissue, or connection of tissue to a prosthetic graft. In this embodiment, the device includes a tube with a suction port completely or partially circumscribing a portion of the distal end. One or more cannulas are arranged so that their distal ends are pointing toward the proximal end of the device and spaced around the circumference of the distal side of the suction port. This configuration is suited for placing a circumferential interrupted suture line for the purpose of anastomosis, prosthesis implantation, defect closure, or outlet reduction. Various numbers of cannulas can be employed. In one variation, to create a single circumferential suture line of simple interrupted sutures, a number of cannulas, for example ten cannula, can be employed. Each cannula can include a single suture needle with a suture attached. The tissue is drawn in and the operator pushes the proximal end of the suture needles into the proximal end of the cannulas. The distal end of the needles penetrate the tissue and continue up the receiving cannulas. Once the distal needle tips reach and exit the proximal end of the main tube, they are grasped by the operator and pulled the rest of the way out of the device. The needles leave the attached sutures incorporated in the tissue. If the desired result is to attach two tissues together to form an anastomosis, after the first tissue has been penetrated by the needles, the needles can be reloaded by pushing them back into the delivery cannulas after the first tissue has left the suction opening. The device is then repositioned so that the second tissue is drawn in to the suction opening and the passing of the needles is repeated, thus incorporating the second tissue. The sutures can then be secured, fastening the first tissue to the second.

FIG. 7 illustrates an extended linear suction port. The width of this embodiment can be, for example, about 10 to 110° of the circumference of the tube, or about 5 mm to about 20 mm. As in other embodiments, the tube may be expandable once in the closed space or hollow organ to enable a larger port in the expanded tube. The length of the port will vary depending upon the application, for example, from about 5 mm to about 250 mm. Once an expandable tube has been inserted into a desired position, the diameter of the distal end of the tube surrounding the suction port may be enlarged to allow for larger tissue bites or to allow for matching the circumference of a lumen or annulus intended for suture incorporation. One mechanism to enlarge the tube is to wrap or fold the tube around itself, similar to a toilet paper roll cut down one side, or a spiral cut. The tube can have two halves that may be extended a distance apart from one another once inside the organ or closed space.

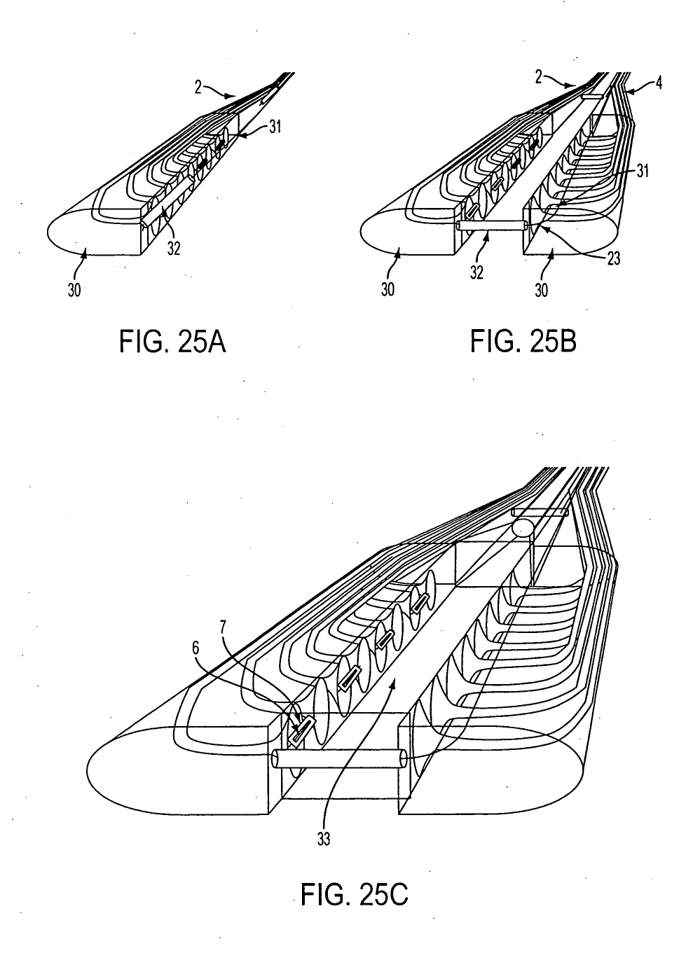

In one embodiment of the linear suturing device, the device is made up of components 30 that are inserted into the organ individually, then assembled inside the organ, as illustrated in FIGS. 25A-C. The use of components allows for easier insertion of small components through a natural orifice, and after assembled, the benefit of a larger device for taking larger, more effective suture bites. The components can be strung together using wire, cables, strings, or sutures 31 so that they can be pulled together and held in place. Spacers 32 positioned on the wire allow components to be held in the proper orientation in relation to each other. After the sutures have been deployed, the device may be disassembled and withdrawn from the patient.

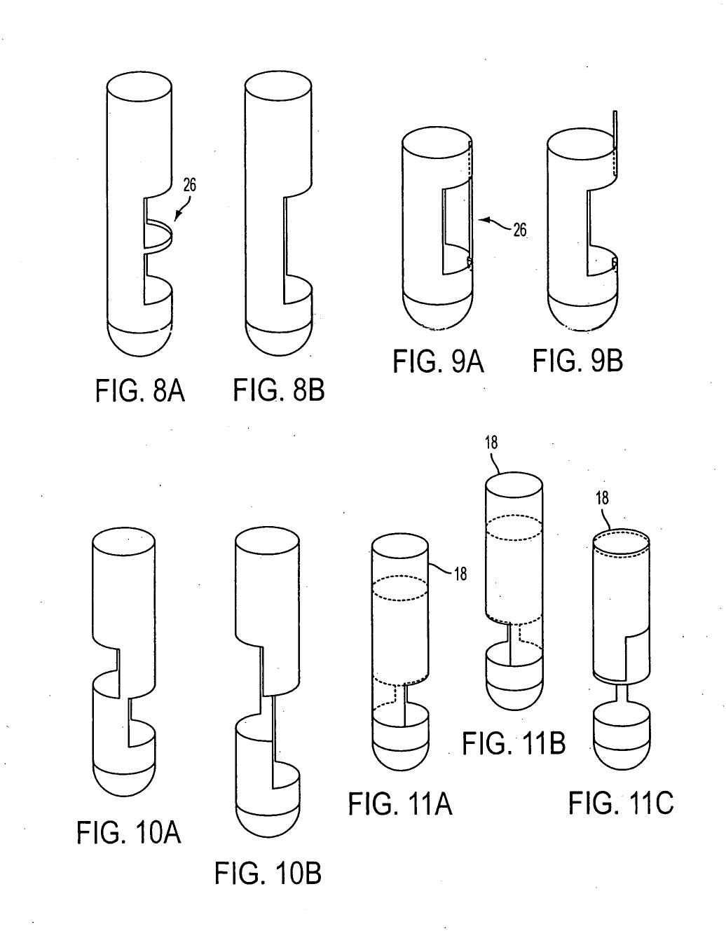

The tubes or capsules can be configured to have multiple suction openings, as shown in FIGS. 8A-11C. All of these multiple suction port configurations can come apart such that the sutures and the incorporated tissue do not become entrapped within the device. The suction ports of these and other embodiments can be opened and closed with internal or external sleeves.

FIGS. 8A and 8B show a configuration of two suction ports positioned vertically on top of one another. The band separating the suction ports can have the ability to be removed from the suction opening by rotating from between the suction ports. This arrangement enables plications, closures and tissue augmentations by passing suture through two tissue bites at one time.

FIGS. 9A and 9B show a configuration in which two side by side suction ports are effectively formed by a single suction port with a removable strut.

FIGS. 10A and 10B show a configuration of two suction openings on apposing sides of the tube. This suction opening configuration could enable means of attaching the tissues drawn into each suction opening to each other. For example, the distal end of a cannula positioned below each opening. A single double-armed suture can be provided with each needle backed into the delivery cannula, thereby leaving a span of suture material running between the cannulas. Suction is applied to both suction openings at the same time, or one at a time by closing the opening with an internal sleeve. The device could be loaded with several double armed sutures in one set of cannula or the device could have multiple cannulas running parallel and arranged and loaded within the tube.

Alternatively, the device could have a delivery cannula positioned above the upper opening so that the needles could penetrate the drawn in tissue on the down stroke, then follow a groove on the bottom of the tube leading to the distal side of the lower suction opening. The needle could continue up through the tissue and up and out of the tube. This results in a simple suture bite connecting two tissue walls. The device could then be reloaded, and the procedure could be repeated, thereby creating a running suture line.

FIGS. 11A, 11B, and 11C show a configuration of a suction port that can switch from one side of the tube to another. Cannulas can run the length of the tube and cross the suction openings at a support strut, then turn or loop at the bottom and be arranged just proximal to the suction openings. The device can, for example, be configured with two delivery portions of cannulas positioned with each delivery portion ending under each suction opening. Alternatively, the device can be configured with multiple cannulas ending under each suction opening. The cannulas can be back-loaded with double-armed sutures so that a span of suture crosses between the opposing cannulas.

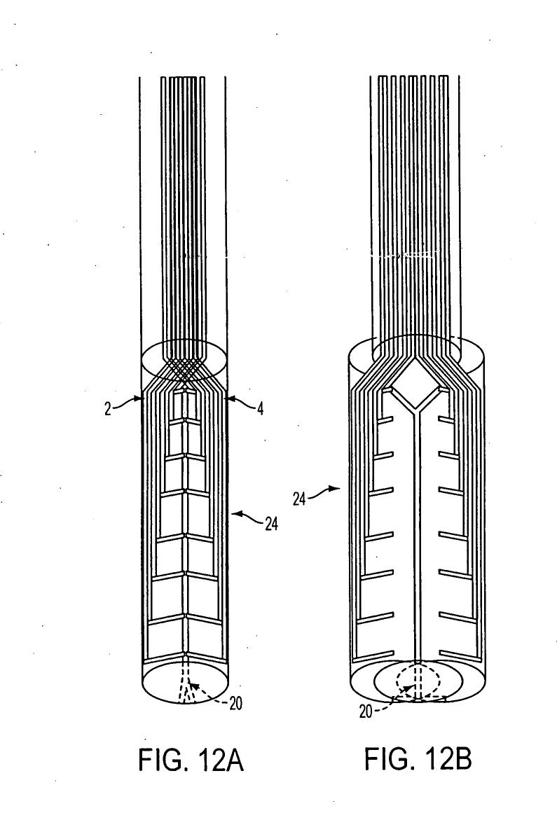

The device shown in FIGS. 12A and 12B is suitable for placing sutures in a linear fashion with cannulas directing the suture needles across a linear suction opening. The device is inserted as shown in FIG. 12A, and then once within the organ or area to be sutured, the device is expanded as shown in FIG. 12B to produce the suction opening. The tube can also have two halves that are connected on one side but not the other, allowing the split side of the tube to have the halves extend away from each other, thus creating a suction port.

The device of the present invention can have one or more cannula, one or more needles, and differing combinations of needles and cannulas to achieve the desired suturing.

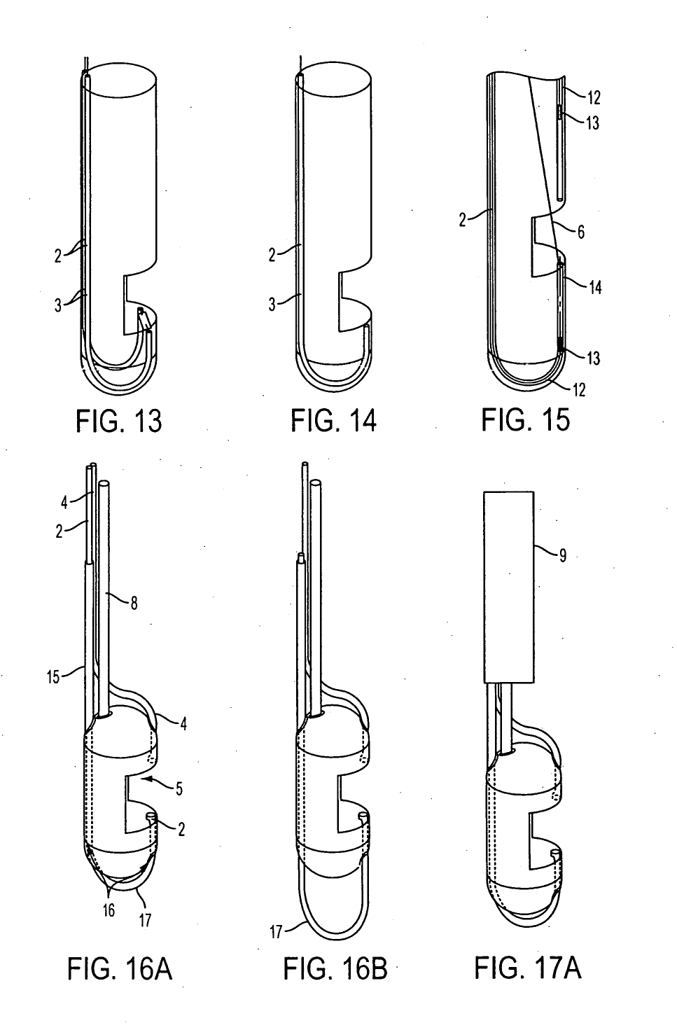

As shown in the embodiment of FIG. 13, the device can have two cannulas 2 for two needles 3. As shown in the embodiment of FIG. 14, the device can have one cannula 2 for one needle 3. The device may have delivery cannula, receiving cannula, both, or neither to direct the needles in the desired locations. In the device shown in FIG. 15, as discussed in further detail below, the cannula can incorporate a push-rod to manipulate a needle that is relatively shorter than the needle described in the other embodiments.

In the embodiment of FIG. 16A, the tube 1 is relatively smaller and capsule shaped. Although the term “capsule” is used, the capsule of the present invention is not strictly capsule shaped. The capsule can be any enclosure with essentially closed ends that defines a suction opening and enables the suction opening to be in fluid communication with the vacuum hose. For example, the capsule can have rounded ends and be slightly elongated, such as the particular capsules shown in FIGS. 16A-B and 17A and B, spherical, cylindrical, or some other shape.

One purpose of the tube is to enable the suction opening to be in fluid communication with the vacuum. The capsule 1 maintains this function while reducing the size of the device. The capsule 1 can have similar attributes as the tube. In this embodiment, the cannula 4 extends from outside of the subject to the capsule 1 The cannula 4 can be enclosed in an outer cannula 15 allowing the inner cannula to move up and down within the outer cannula. A vacuum hose 8 provides the vacuum for the suction opening. The cannula 4 can be incorporate a segment of support material that can be affixed to an endoscope. In this embodiment, the cannula are of a sufficient length to extend from the area of insertion to the suture location.

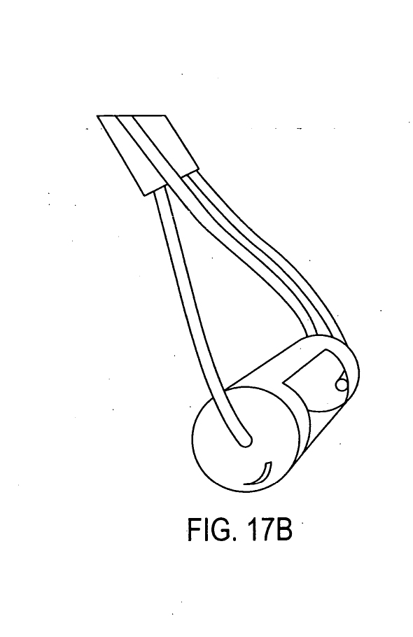

In the embodiment shown in FIGS. 16A and B and 17A and B, the capsule 1 is arranged to place vertical sutures. Alternatively, the capsule can be arranged to place horizontal sutures by pivoting the capsule in relation to the attached endoscope. In fact, by rotating or pivoting the capsule 1 as illustrated in FIG. 17B, the sutures can be placed in any direction, for example, diagonally. This feature is accentuated by the flexibility of the cannula 4, vacuum hose 8, and needles.

The devices in FIGS. 16A and B and 17A and B have a single cannula, although additional cannulas can be provided. When the single cannula embodiment is loaded with a double-armed suture, the device can provide mattress suture bites.

It can be difficult to push the end of a needle (proximal or distal end) through the 180 degree loop of a cannula at the bottom of the tube or capsule. Therefore, in one embodiment the cannula system allows for this and obviates the need to push the needle around the loop. This is done by enabling the flexible cannula to drop down either within the distal end of the tube, or beyond the distal end of the tube as illustrated in FIG. 16B. By dropping down, the needle can be passed within the cannula to a point in distance past the length of the loop. Once the end of the needle is past this point, the cannula can be pulled up to its original position, bending the enclosed needle as this is done. At this point, the needle has essentially made the turn and can now be easily pushed the remainder of the way through the cannula system. This feature can also be incorporated into the other embodiments.

The embodiment of FIG. 17A is particularly useful for back loading a needle into the delivery portion of the cannula. After the needle has passed through the drawn in tissue and cannula system, the needle can be flipped and sent, tip end first, down the receiving portion of the cannula to take a subsequent suture bite in a second drawn in tissue. This arrangement enables a second bite to be taken on the down stroke, thereby enabling a user to alternatively sew back and forth through the cannula system.

As shown in FIG. 16C, this embodiment can include an endoscope 9. The cannula can also be incorporated into an endoscope. If an endoscope is provided, the endoscope can include an instrument channel, and the cannula can be arranged within the instrument channel. Alternatively, the cannula can run along the side of the endoscope.

Because tissue lumens can be of varying sizes, the device can have the ability to expand and contract its diameter at and around the suction opening. In one such embodiment shown in FIGS. 18A and 18B, a series of rigid cannula 21 segments are attached or connected to each other, radially spaced about the circumference of the tube, by a flexible plastic, fabric, or cellophane material 22. These rigid cannula sections 21 could be made of a material such as steal or plastic. These rigid cannula sections 21 could be attached to struts 20 that extend and retract similar to the struts of an umbrella when the fulcrum points of their attachments are slid near each other on a shaft, for example, in area 24. In the device, when the struts of the “umbrella” are extended, the flexible plastic, fabric, or cellophane material will make up the wall of the tube or capsule and form a suction port 19. When the struts of the “umbrella” are collapsed, the flexible plastic, fabric, or cellophane material “accordions” down to size. The diameter of the tube near the suction port may have the ability to expand in a range from approximately 10 mm to 120 mm. A circular cutter could be incorporated into the device to cut out unwanted tissue from the tissue lumen. This circular blade may be positioned in the proximal tubular section above the suction port, in a distal end cap section below the suction port, or both. The circular blade can have slits cut in the circumference to allow for the support struts of the suction port.

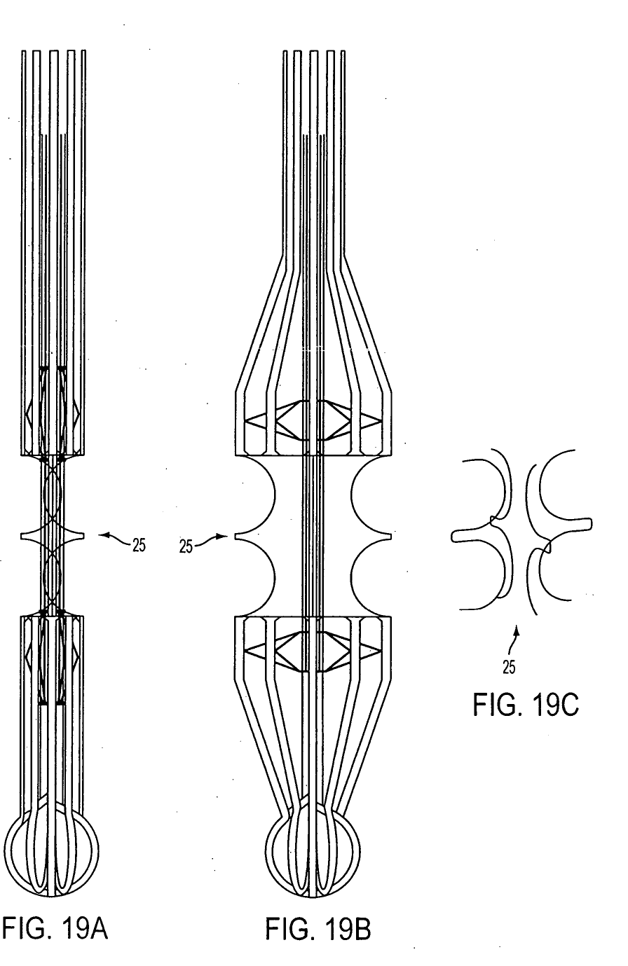

FIGS. 19A-C illustrate an embodiment similar to the embodiment of FIGS. 18A and 18B, except that the device includes two suction ports. The two suction ports are supported by a support cage 25.

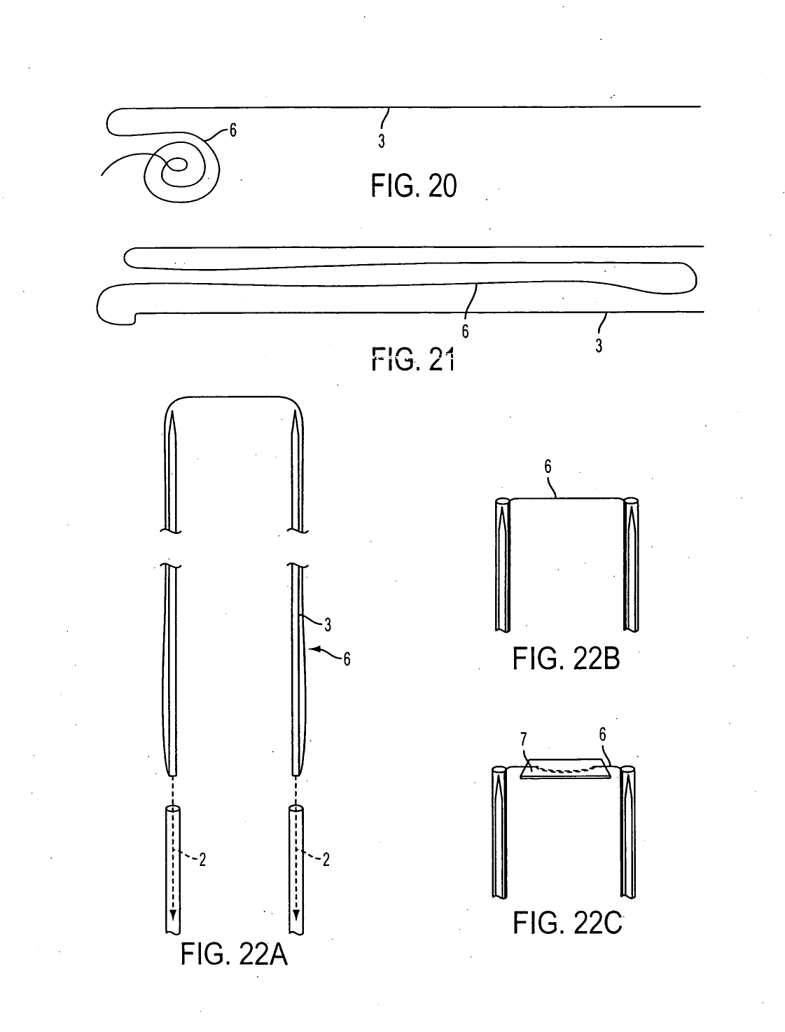

FIG. 20 shows a single armed suture embodiment with one needle attached to one suture In the double armed suture embodiment shown in FIG. 21, two needles are attached to either ends of the same suture. The suture may incorporate a pledget or similar device to help prevent the suture from tearing through the tissue. The suture may incorporate a fastener such as a “self-tying U-clip” device that can be pulled into the desired position by the suture. The U-clip device can also be made of Nitinol. The U-clip devices are available in multiple sizes and lengths to facilitate the operator's choice of surgical technique.

FIGS. 22A-C show one way the needles can be loaded into the cannula. In this arrangement, a double armed suture is back loaded into two cannulas, leaving a span of suture material 6 between the cannulas. A pledget 7 can be added.

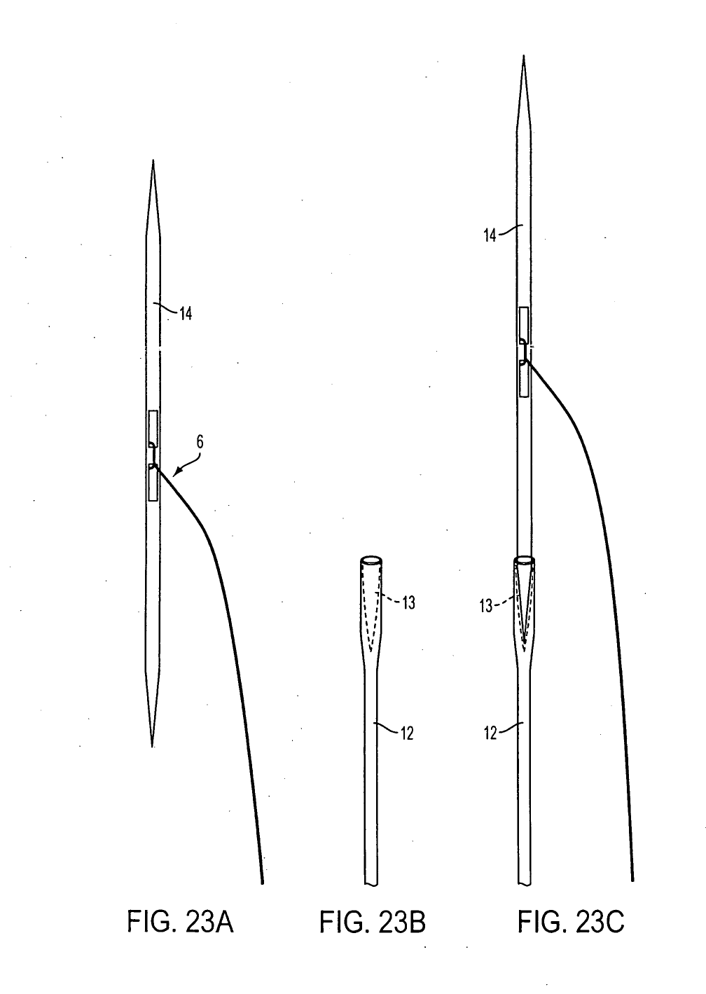

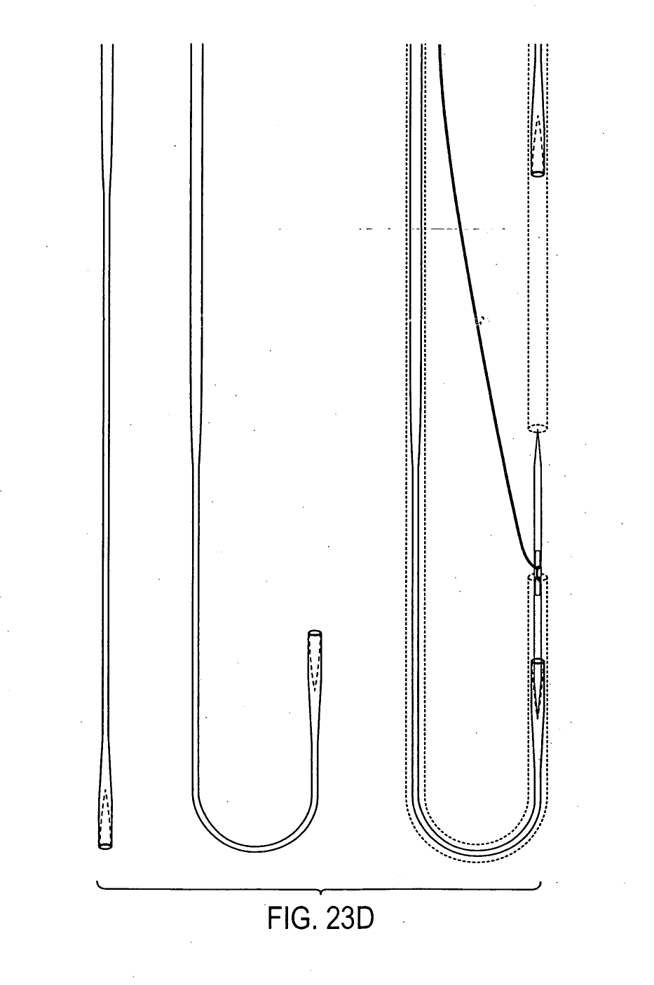

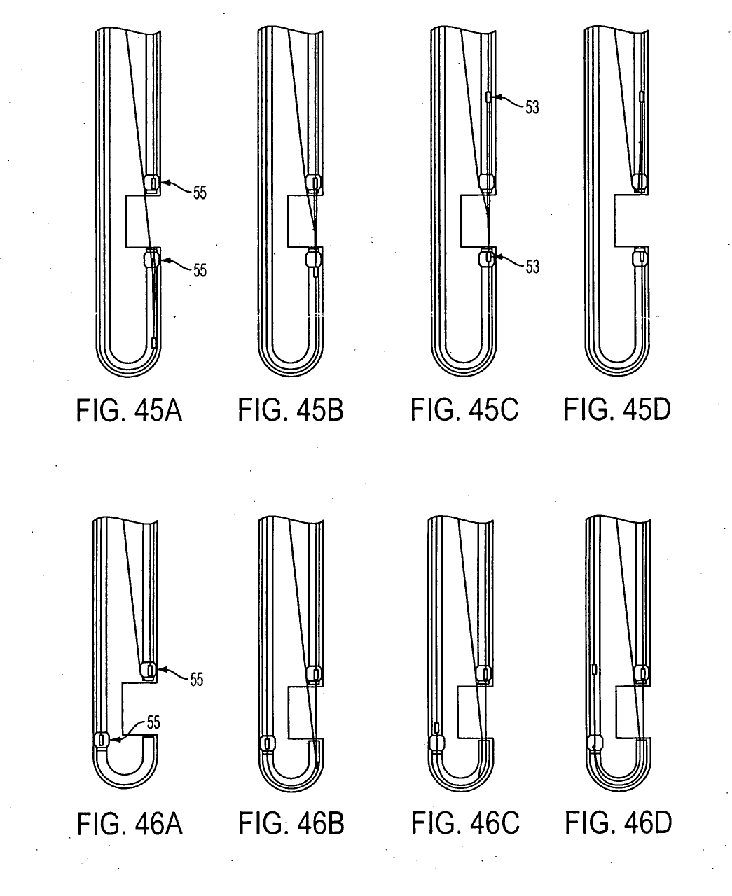

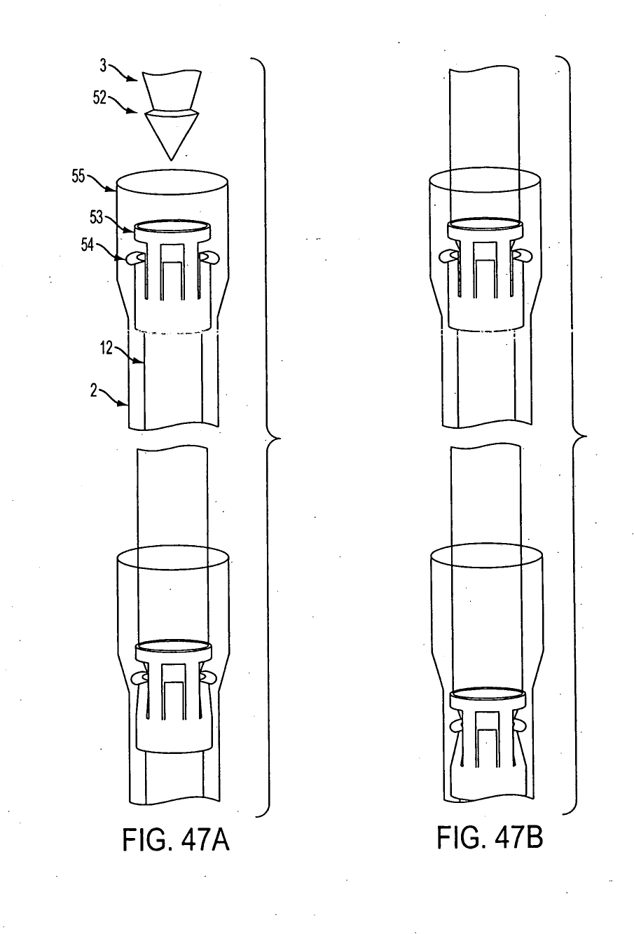

FIGS. 23A-23D show yet another needle embodiment. This embodiment is particularly useful for the device shown in FIG. 15, FIGS. 45A-D, and FIGS. 46A-D and includes relatively shorter needle 14 with a point on each end. The suture material 6 is connected to the needle 14 at approximately a mid-point. A push-rod 12 can be used to manipulate the needle by pushing the needle with a receiving cavity 13.

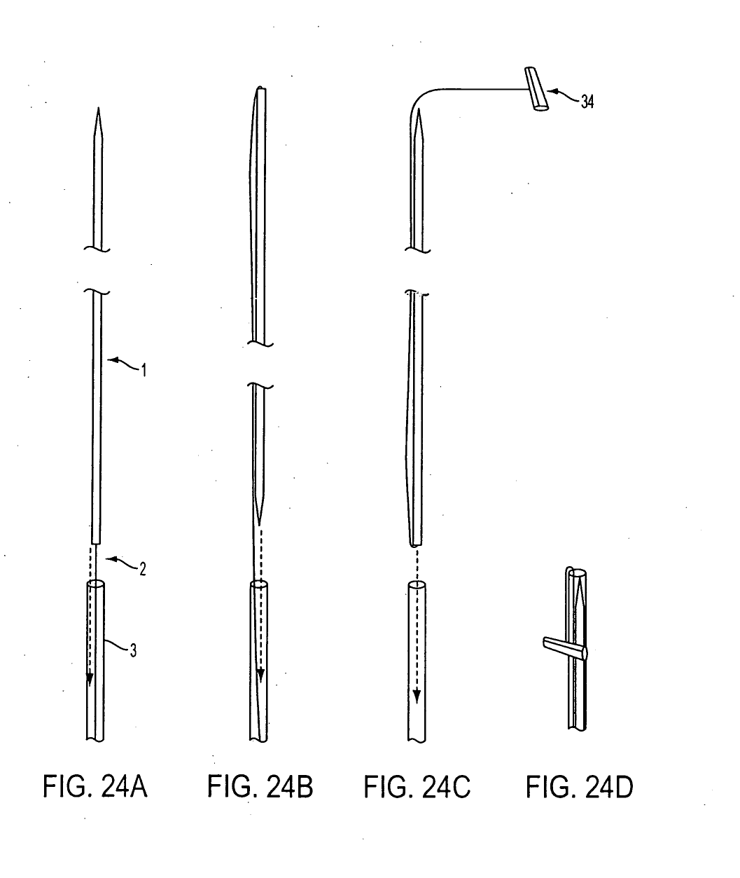

FIGS. 24A-D show another way the needles can be loaded into a cannula. In this arrangement, a single armed suture is back loaded into a cannula and includes a suture anchor 34. In order to take multiple bites within the organ, vessel, or closed space, the device has the ability to reload the needles after each bite. The device of the present invention may employ various methods to reload the needles.

In one reloading embodiment, a needle is reloaded by pushing the needle back into the delivery cannula after each suture bite. This is facilitated by having a reloading or receiving cannula on the proximal side of the suction port. The receiving portion cannula can be removable from the main tube. In this reloading procedure, the needle tips pass through the tissue and emerge on the proximal side of the drawn-in tissue. The needles enter a receiving cannula and continue up the distance of the tube. The operator grasps the distal end of the needles, pulling the needles the full length of the receiving cannula. This creates a first tissue bite or piercing of tissue. The vacuum is deactivated. The tube may then be backed or turned away from the recent suture bite to allow the drawn-in tissue to be released from the bore of the tube. The receiving cannula can now be used to reload the device to suture a second tissue. In one embodiment, the receiving cannula can be releasably held within the tube and lowered directly over the delivery cannula by sliding down and traversing the suction port. The needle may then be pushed back into the delivery cannula with the slack suture riding along with the needle within the cannula. Once the proximal end of the suture needle emerges from the proximal end of the delivery cannula, the needle is grasped and pulled until the needle tip is again aligned with the distal end of the delivery cannula. The receiving cannula is slid back to its original position and resumes its original function of receiving the needle from the delivery cannula. The slack in the suture can be managed by pulling on the suture loop at the proximal end of the delivery cannula. Now, the device is ready for a second suturing procedure. The vacuum can be applied again to draw in another portion of tissue, and the needle is ready to be pushed through the tissue to deliver the suture. This process may be performed as many times as desired, thereby allowing for the tying together of multiple bites of tissue. Once the final suture bite has been taken, the receiving cannula can be removed from the tube, allowing the sutures to be secured to one another. This embodiment can also utilize various combinations of multiple cannulas and multiple needles to result in any desired resulting suture arrangement.

In another embodiment, the needle may be reloaded by using the receiving cannula as the delivery cannula and the delivery cannula as the receiving cannula. In this embodiment, after the needle has passed through the first tissue bite and has been pulled out from the proximal end of the receiving cannula, the needle is flipped and reinserted, tip first, into the receiving cannula. With a subsequent tissue drawn into the tube, the needle is pushed through the tissue and back into and through the delivery cannula. This process could continue to enable the creation of a continuous suture line by alternately sending the needle up and down the delivery and receiving cannula. The openings of the cannulas proximal and distal to the suction opening may be flaired or trumpet shaped to ease the task of pushing a needle into the cannula.

In another embodiment, the cannulas may be removed from the main tube and reloaded outside the tube. The cannula or cannulas, now loaded with suture needles, can then be reinserted into the main tube.

In the embodiment shown in FIGS. 25A-C, the device is essentially assembled in the patient. The device includes a cannula delivery component 31 that includes a series of delivery components. A wire 31 and spacer 32 extend from one side of the cannula delivery component 31. A receiving component is slid down the wire 31 and spaced apart from the delivery component by spacer 32, as shown in the position of FIG. 25B. A vacuum component containing a third wall is manipulated in between the delivery and receiving components for forming a vacuum to attract tissue in between the delivery and receiving components.

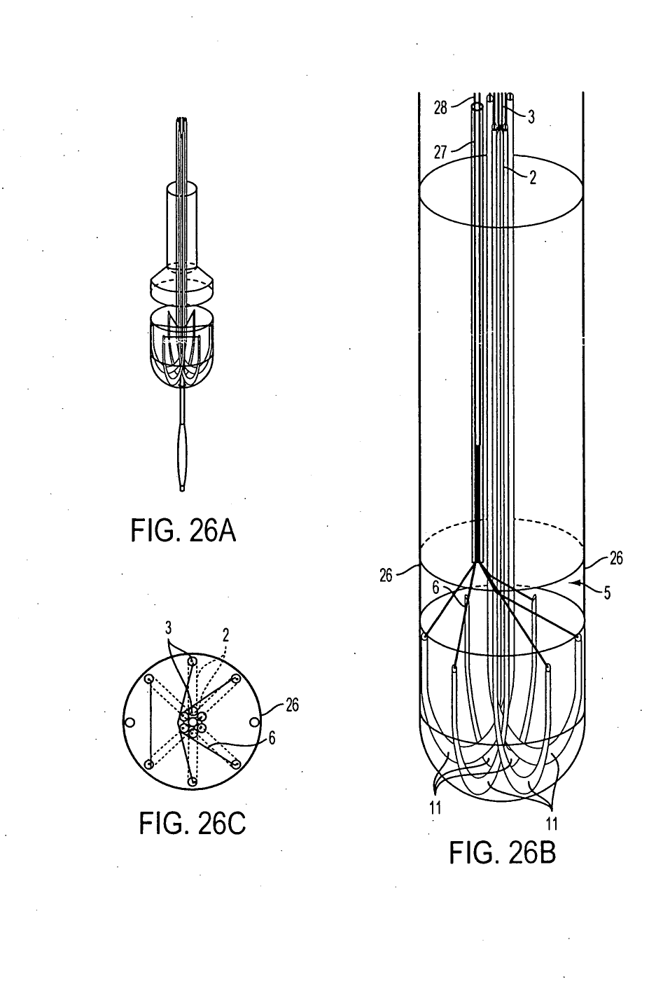

The embodiment shown in FIGS. 26A-C is especially suited for closing defects or reducing the size of an orifice or outlet. In the particular arrangement of FIG. 26A, each of the delivery portions of the cannulas are associated with a single return portion of a single cannula. FIG. 26B demonstrates a variation of the device of FIG. 26A for placing the sutures on apposing sides of a defect. A retractable strut 26 spans between the top portion and bottom portion of the tube 1. The device can also include a suture maintenance cannula 27 and suture maintenance string 28. FIG. 26C is a cross-sectional view of the suction opening area and the configuration of sutures loaded such that the double-armed sutures are loaded such that the double armed sutures span across the device between the cannulas containing their connected suture needles. In this example, the suture spans are loaded such that all the suture spans are located to one side of the central shaft which is made up of the delivery cannula.

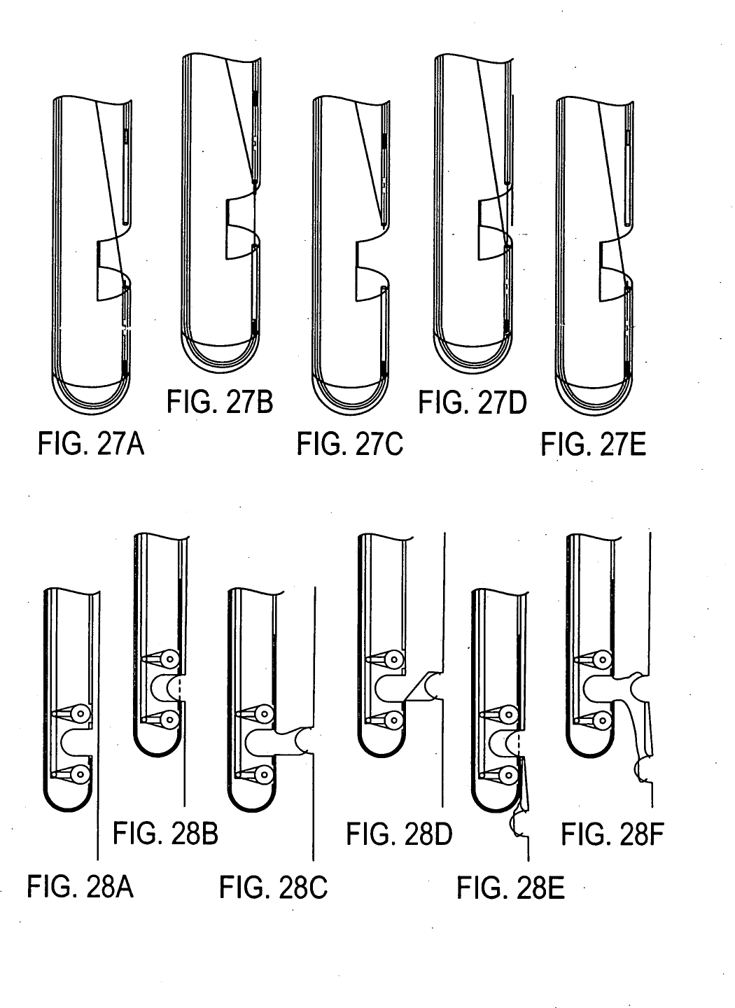

A further embodiment is illustrated in FIGS. 27A-E, and utilizes a relatively shorter needle. In the position shown in FIG. 27A, the needle is positioned on the distal end of a suction port. As shown in FIG. 27B, the needle is pushed by a rod across the suction port and, in use, through tissue. The rod disengages the needle and retracts back across the suction port, leaving the needle on the distal end of the suction port, as shown in FIG. 27B. As shown in FIGS. 27C and 27D, a second rod can engage the needle and push the needle back across the suction port. The second rod can then disengage from the needle and retract back across the suction, leaving into the needle in the original position, as shown in FIG. 24E.

As illustrated in FIGS. 47A-B, the needle that is designed to be shuttled back and forth across a suction opening may incorporate a barb 52 that can be caught and held by a designed catch on the end of the pushrod 53. This will enable the needle to be pushed a portion of its length across the suction opening until it enters the receiving cannula and is engaged by the catch on the end of the opposing pushrod. The opposing pushrod having grasped the end of the needle can now pull the needle the remainder of its length across the suction opening.

The embodiment in FIGS. 47A-B demonstrate how the catch 53 is able to hold or release the barbed needle tip by moving the catch in or out of an area of the cannula with an increased diameter 55. The catch may incorporate flanges 54 that can be bent in or out of the catch assembly. FIGS. 47A-B illustrate a series of flanges that are bent out. When the catch is in the portion of the cannula with an increased diameter, the flanges are held out of the engagement position, as shown in FIG. 47A. FIGS. 47A-B illustrate how the flanges are forced inward and hold the needle tip as the catch is pulled or pushed into the narrower cannula section.

As briefly mentioned above, the needle and push-rod shown in FIGS. 23A-C are useful for this embodiment. The needle can be sharpened on each end and be attached to a suture, for example, in the middle of the needle. Nitinol wire, or similar material, can be used as the rods to push the needle. The rods have a cup or catch to engage the needle tip. In this embodiment, the needle can go through the tissue in either direction, or it can be reloaded by pushing the needle back from which it came and then the needles can go through subsequent tissue traveling in the same direction.

As shown in FIG. 23D, the push-rods can have a tapered, or a relatively thinner, portion in the area of the bend of the cannula. This enables the push-rod to turn easier in the bend because the tapered portion is more flexible, while the rest of the thicker portions of the push-rod maintain the strength necessary to manipulate the needle. The length of the tapered portion is approximately the length that the needle must travel across the suction port to be received by the opposing push-rod.

FIGS. 45A-D illustrate how a needle designed with barbs on each of its sharpened ends is shuttled across a suction opening. FIGS. 45B and 45C illustrate how one end of the needle is maintained in a catch as the other end is released with only one catch 53, of the two pushrods, within an expanded diameter section of cannula 55 at one time.

FIGS. 46A-D illustrate how a flexible shape memory needle designed with barbs at each end of its sharpened ends could be shuttled across a suction opening. In this embodiment, the pushrods remain relatively straight as the needle bends around the distal end of the tube. This will allow the length of the tube or enclosure distal to the suction opening to be shortened.

As alternatives to the manual advancement and reloading of the suture needles, other embodiments of needle propulsion are provided. As shown in FIGS. 28A-F, the device may employ cylindrical or round rollers that are positioned proximal and distal to the suction port. The rollers are positioned to apply pressure on a needle coming between the roller and the wall of the tube or between two rollers within the tube. The rollers can be controlled to spin in either direction and can be made of a material that grips, for example, a rubber or elastomer. A needle is positioned within a cannula system that has breaks in it to allow for the rollers and the suction port. The distal roller may rotate and propel the upward transecting the plane of the suction port. The needle would be long enough to reach and become engaged by the second roller. The second or proximal roller pulls the needle completely through the area of the suction port. The proximal and distal rollers then have the ability, after the device was disengaged from the tissue, to spin or roll back the needle to its original starting position within the distal cannula.

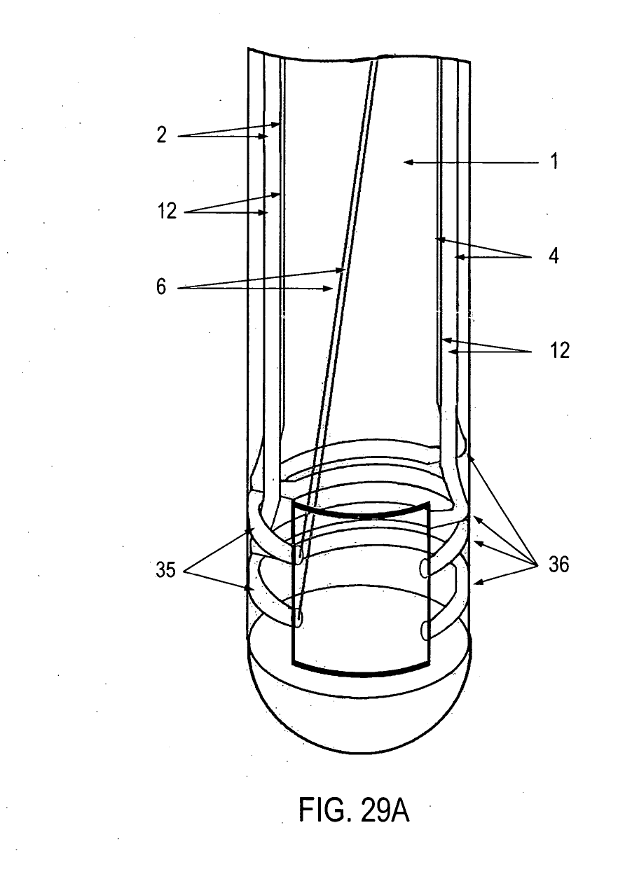

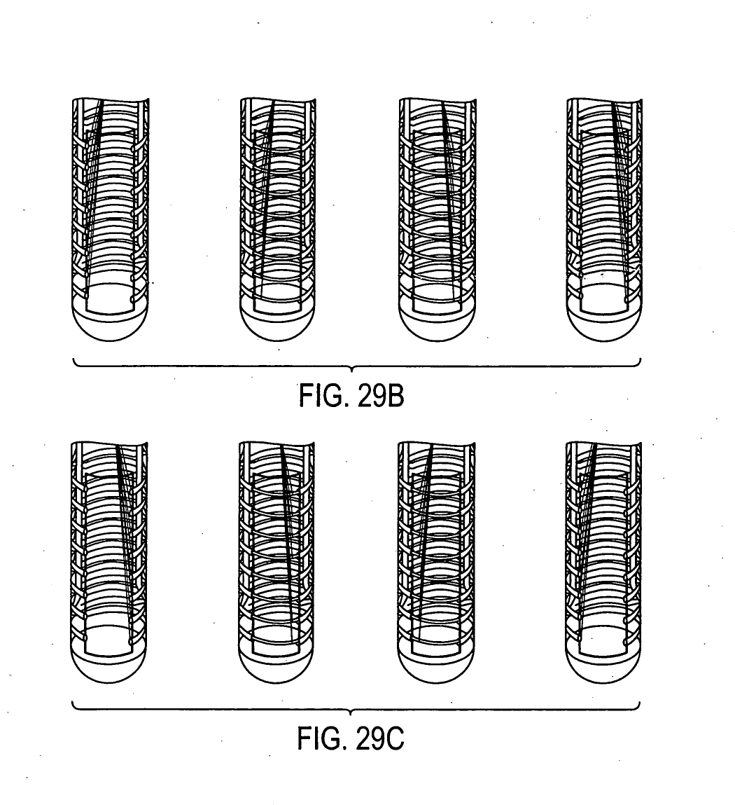

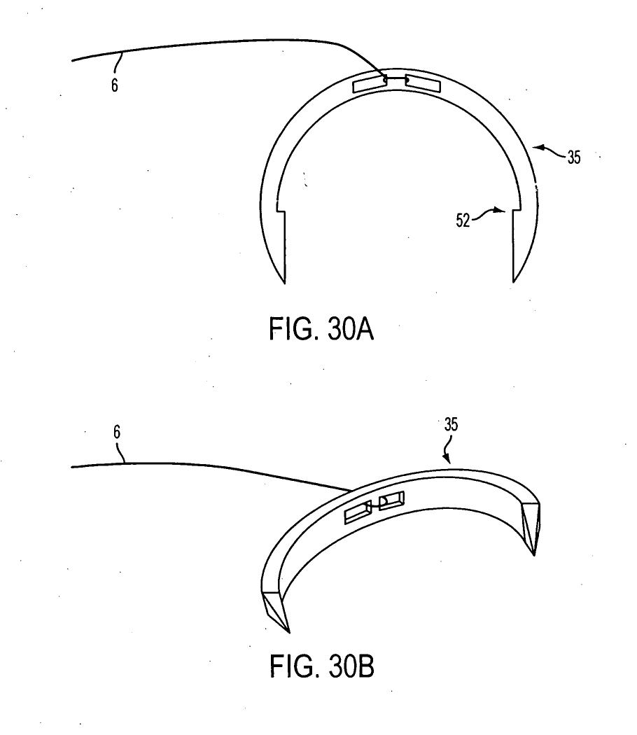

An variation of the embodiment shown in FIGS. 27A-E is illustrated in FIG. 29A. In this embodiment, the cannulas bend such that a curved needle, FIGS. 30A-B, can be passed horizontally across the suction port. In one use of this embodiment, an operator can send the needle though one wall of tissue, then move the device to a second wall of tissue, and then push the needles back through the other way. The device could then be withdrawn with the suture slack trailing to be secured. FIGS. 29B-C show a variation of the device shown in FIG. 29A. In the far left side of FIG. 29B the needles are ready for suturing. The push rods push the needles across the suction opening. The needles are caught by the apposing pushrods and pulled the remaining distance across the suction opening. Then, opposing push rods push the needles back across the suction openings. Finally, the opposing push rods catch the needle tip and pull the needle the remaining distance across the suction opening, leaving the needles back in the starting arrangement.

The needles can be single armed or double armed. The sutures can be maintained by running up the length of the device and managed, for example, manually for tension and organization. Or, the suture can be maintained on a spool within the device. The spool can, for example, be dropped out of the suction opening into, for example, the stomach, enabling it to unwind and release the suture as the device is withdrawn from the patient, pulling up the slack as it withdrawn.

In this embodiment, the cannulas are fixed in position so that the needle can be passed in each direction successfully. This is achieved by aligning the delivery and receiving cannula in a slight spiral 36. The device can be configured such that the tube is rigid in areas where the distal section of cannulas are placed, and/or have a flexible portion between these sections enabling the area of tube encompassing the suction opening to bend while maintaining the geometry of the opposing delivery and receiving cannulas.

In all the embodiments where pushrods are utilized to shuttle a needle across a suction port, these pushrods could be replaced by the use of hydraulics or pneumatics to move a needle catch 53 back and forth within a cannula.

As shown in FIGS. 30A and 30B, the needle utilized in this embodiment can be semi-circular or non-straight. The needle can have a non-circular cross-section, for example, a rectangular cross-section.

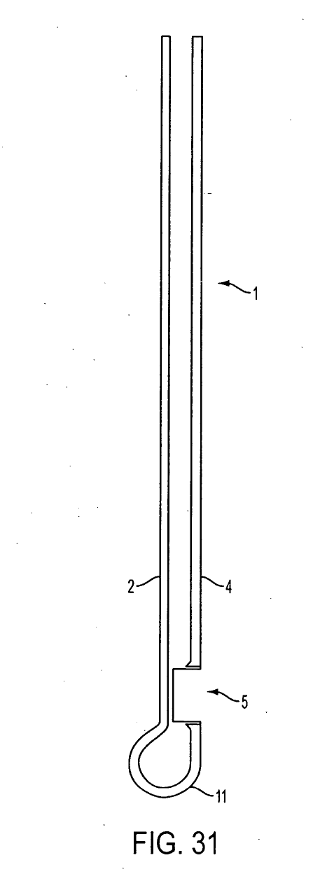

The embodiment shown in FIG. 31 is a generally smaller diameter device for vascular and/or urilogical applications.

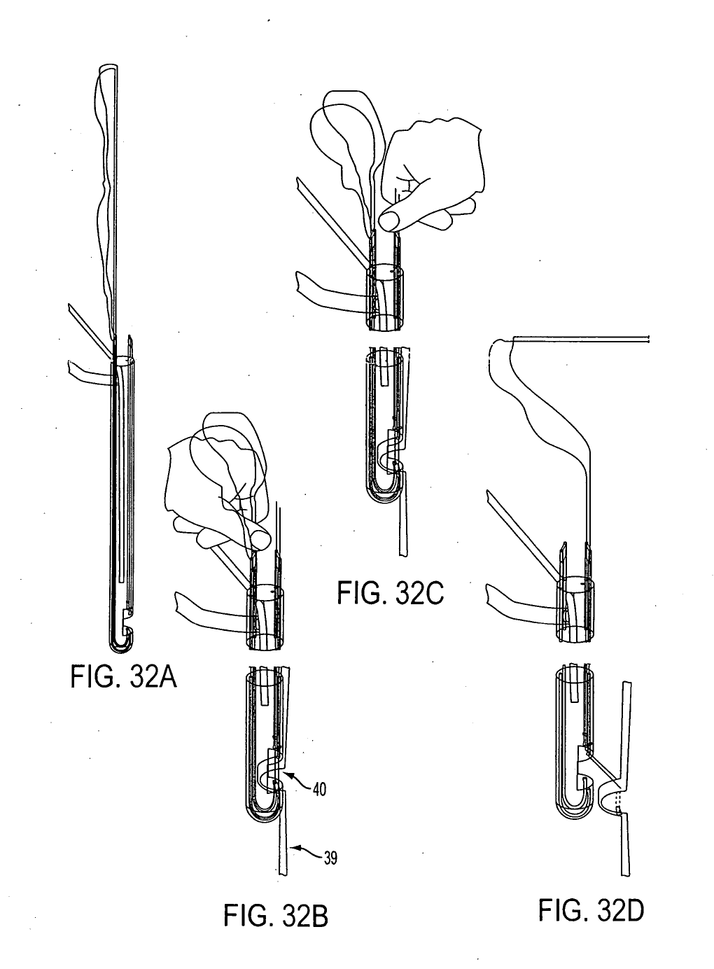

As shown in FIGS. 32A-D, in this embodiment, the device can be used to form a tissue bite or plication in an organ wall. One double-armed suture, consisting of two suture needles attached to one another by a length of suture material back-loaded into separate cannulas. The needles can be, for example, six feet long. The attached suture rides next to the needles within the cannulas. The needle tips are positioned at the distal end of the cannula. The loop of suture connecting the needles now loops out of the distal ends of each cannula and results as a span of suture bridged between the two distal ends of the cannula within the tube. Optionally, this span of suture could have a pledget on it.

The vacuum is activated and the tissue 39 is drawn-in to the bore of the tube. The needles are pushed through the cannulas, penetrating the tissue, and then continue up the main tube. The distal ends of the needles are grasped and the needles are drawn completely through the drawn-in tissue 39. The vacuum is deactivated. The suture can now be tied or secured to produce a single mattress suture bite. Pledgets can be incorporated on either or both sides of the tissue.

If the desired effect is to connect two areas of tissue, the device can be reloaded with the same double-armed suture and repositioned with the suction port apposed to the desired tissue for the second suture bite. The vacuum is reactivated and tissue is drawn into the bore of the tube. The needles are again pushed through the cannulas, penetrating the tissue, and continue up the main tube. The distal ends of the needles are again grasped and are drawn completely through the drawn-in tissue. The attached suture would thereby be incorporated into two tissue bites. The suture can be tied or secured. If desired, additional suture bites can be preformed by continuing to reload the device with the same double-armed suture, creating a double running suture line. Alternatively, the device could be reloaded (backloaded) with a new double-armed suture needle after each bite, creating a line of interrupted mattress suture bites.

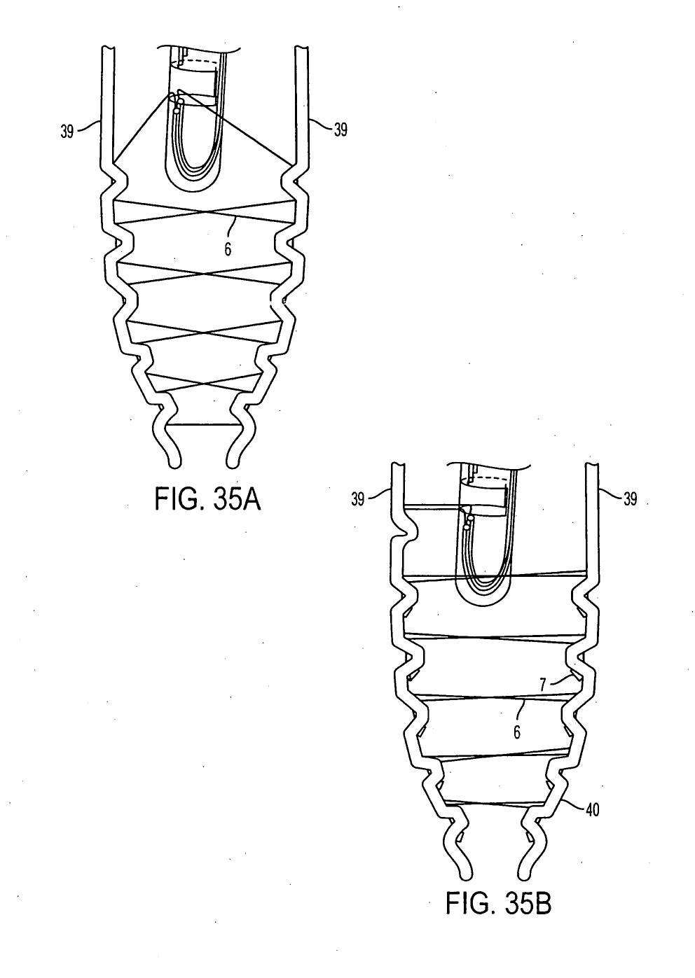

FIGS. 35A and 35B illustrate how a device configured with two delivery cannula could utilize each of the two needles independently to effectively lace together two walls tissue.

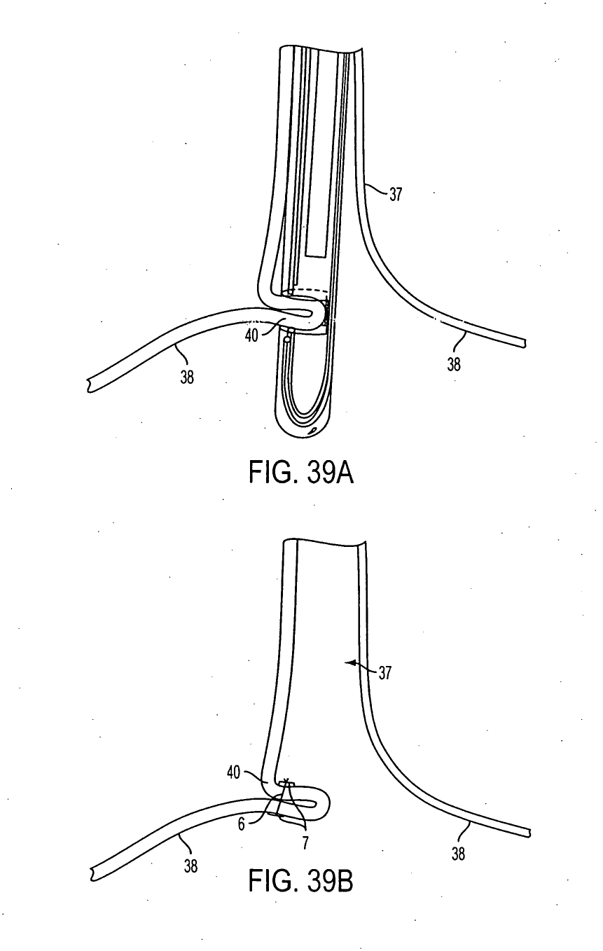

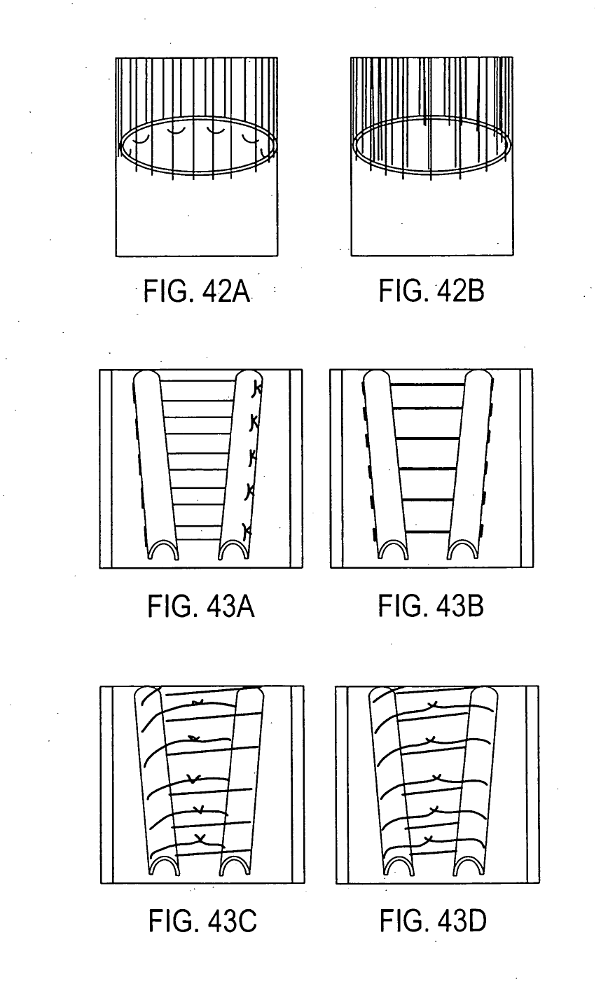

FIGS. 39A and 39B show the device used in the stomach 38 below the esphogus 37, and FIGS. 42A-H show other suturing configurations possible with this embodiment of the device in accordance with the present invention.

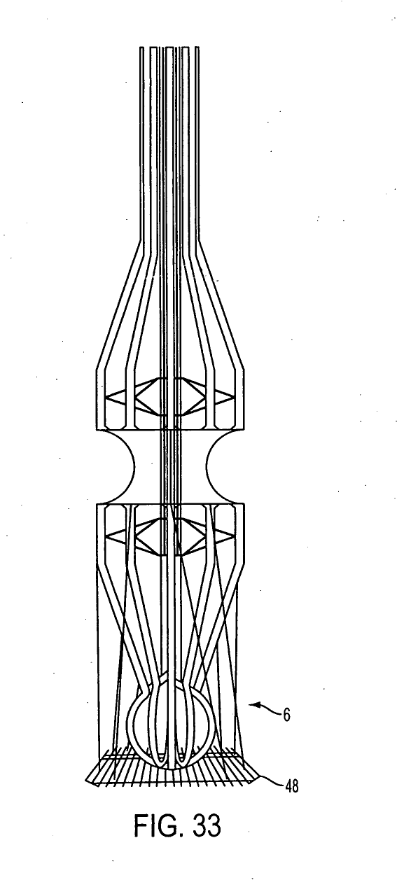

FIG. 33 shows an expandable anastomotic device, for example, the device shown in FIGS. 18A and 18B with a prosthetic graft or patch 48 loaded on the end of the device, incorporated with sutures and ready for implantation at the targeted surgical site.

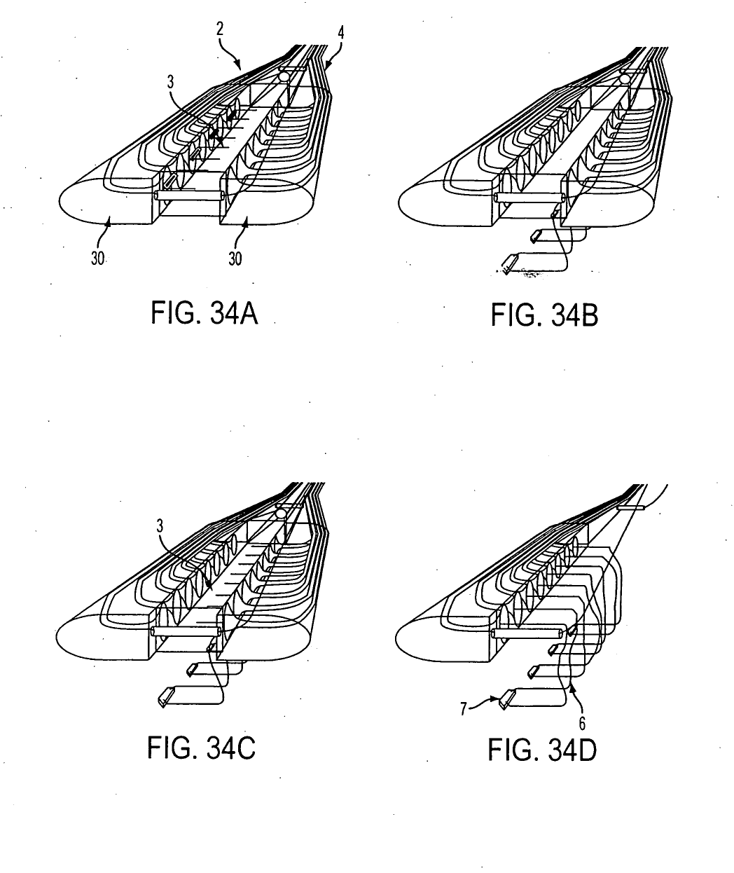

FIGS. 34A-D show an expandable device, for example, the device shown in FIGS. 25A-C placing a line of sutures 6 with pledgets 7.

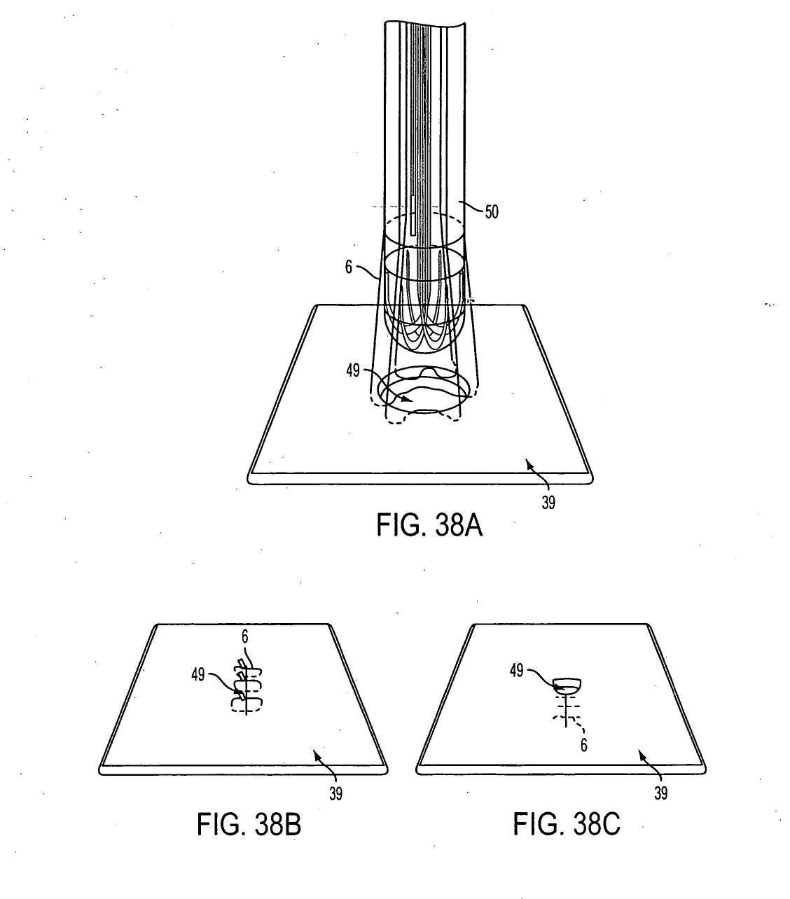

FIGS. 38A-C show the device of the present invention, for example, the device shown in FIG. 6 placing sutures to close a defect, incision, or outlet in an organ wall 39. FIG. 38C shows sutures used to reduce the size of a defect, incision, or outlet.

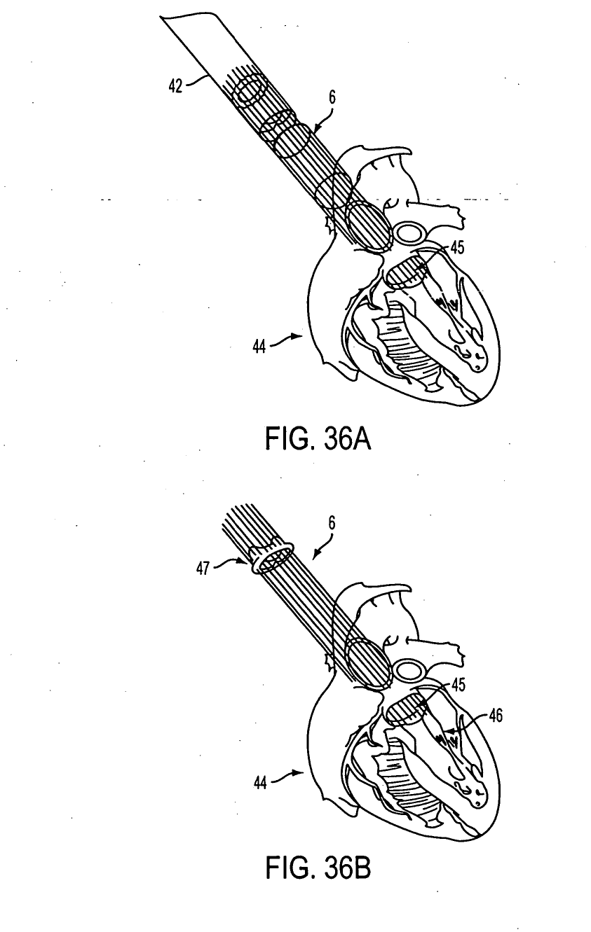

FIGS. 36A and 36B show the device of the present invention placing sutures to connect a prosthetic valve 47 in the aortic annulus of the heart 44.

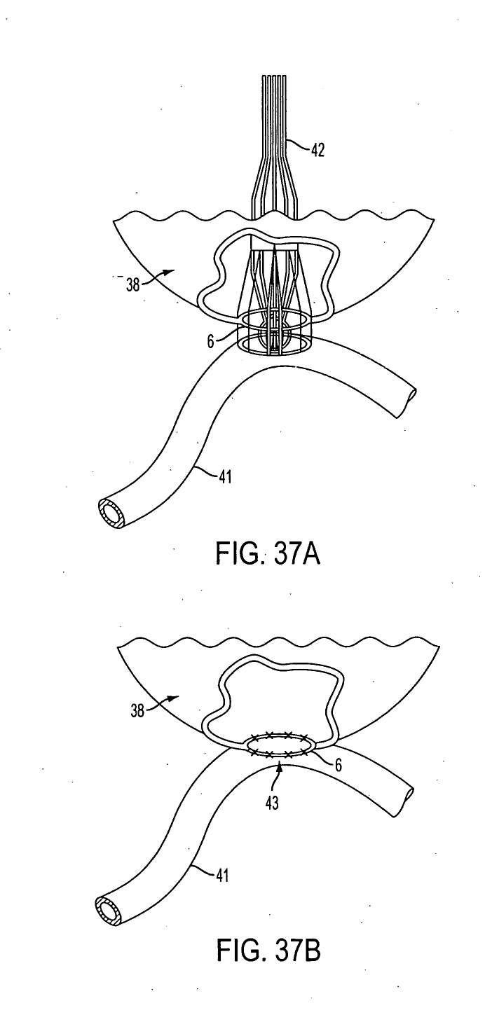

FIGS. 37A and 37B show the device of the present invention placing sutures in an anastomtic pattern to connect the bowel to the stomach.

FIGS. 35A and 35B show two examples of the present invention placing sutures to connect two organ walls.

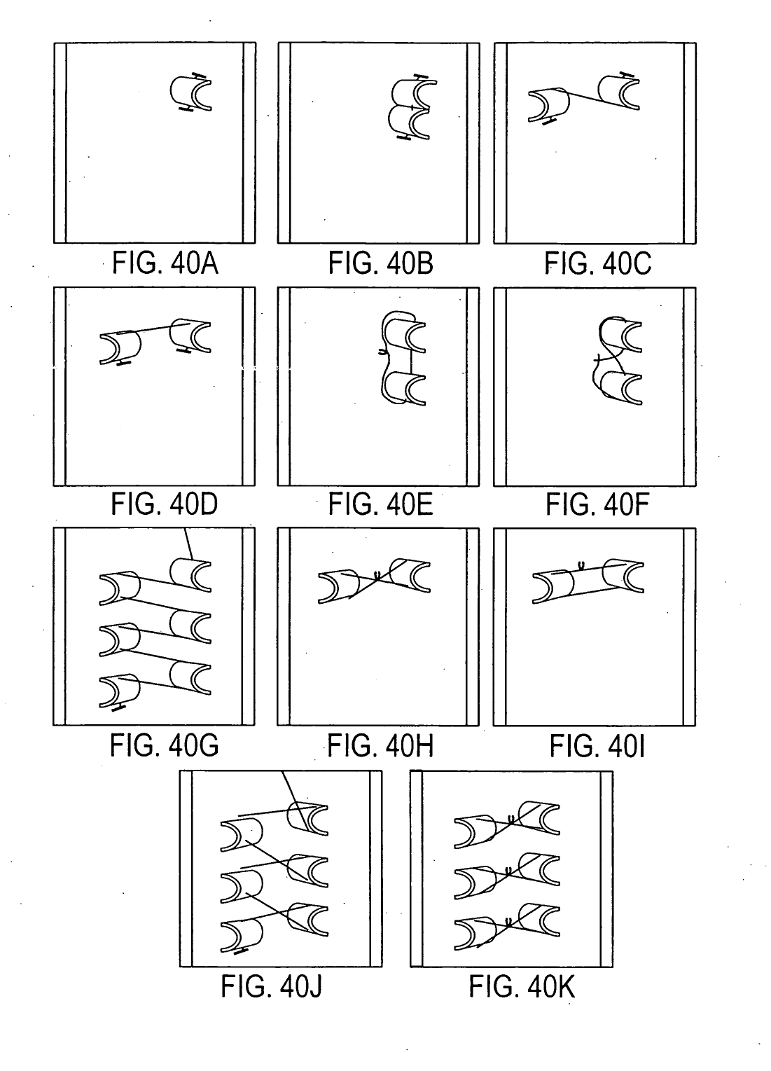

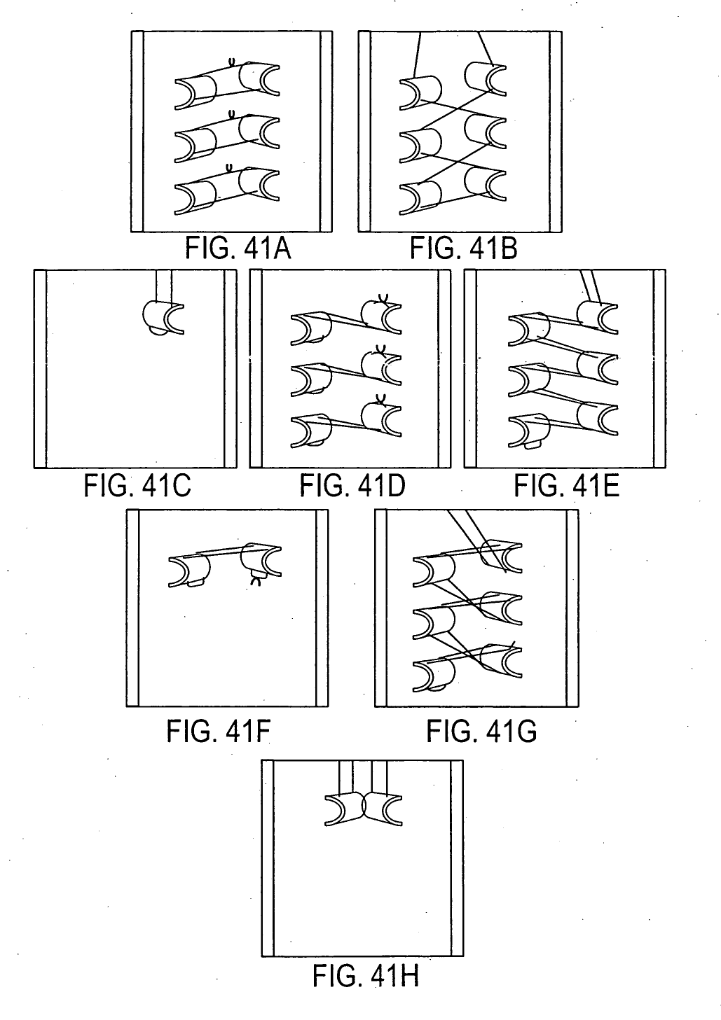

FIGS. 40A-K and 41A-H show various suture arrangements that can be placed with the device of the present invention.

FIGS. 42A and 42B show examples of circumferential suturing that can result from the device of the present invention, for example, the embodiment shown in FIG. 18B. FIG. 42A shows a single circumferential suture line of interrupted mattress suture bites. FIG. 42B shows a line of circumferential simple interrupted suture bites.

FIGS. 43A-D show examples of possible suturing configurations using a linear suturing device configuration of the present invention, such as the embodiment in FIGS. 12A, 12B and 29A-C.

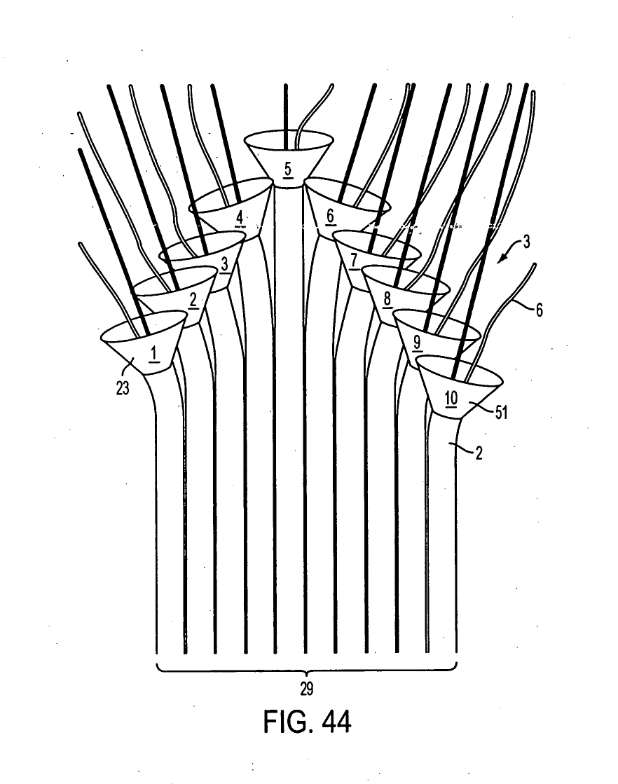

As shown in the embodiment of FIG. 44, the needles and/or sutures can have color codes or markings 51 to assist in managing the needles and sutures within and outside of the device. It can be important to tie or secure the appropriate suture arms to one another. A suture holding system on a dedicated instrument table in the procedure room may help with suture and suture needle management. FIG. 44 also shows that the cannula can have flared or trumpet shaped openings, and can be arranged in a connected cannula ribbon.

In another embodiment, the tips of the cannulas have the ability to shift within the tube. This shift may move the series of laterally up within the tube a short distance, approximately 1 cm. This shift would allow the cannulas, each loaded with a double-armed suture, to engage the tissue with the first arm and then shift, allowing the second arm to be placed parallel to the path its mate followed. This process will create a mattress suture bite.

The suture may incorporate an anchor at one end. The anchor shown in FIGS. 24C-D can be, for example, a small bar perpendicular to the length of the suture that forms a “T” shape with the suture such that the suture cannot be completely pulled through and out of the tissue, thereby anchoring the distal end of the suture line. The T-shaped anchor could be able to traverse the cannula by traveling through the cannula on its short axis. Anchors with other shapes can also be utilized. Anchors can be utilized by having the suture needle back-loaded into the cannula so that anchor is left outside the cannula within the main tube as illustrated in FIG. 24D.

The device can be used in multiple surgical specialties. These specialties may be, but are not limited to, gastrointestinal surgery, cardiac and vascular surgery, gynecological surgery, pulmonary surgery, and general surgery, and may include procedures such as endoluminal gastroesophageal reflux disease procedures such as augmentation of the gastric cardia, gastrointestinal surgery such as gastric reduction or gastroplasty, gastric bypass or gastrojejenoscopy, intestinal anastomosis, gastric excision procedures, outlet reduction, control of gastric bleeding, gastric closure following transgastric surgeries, cardiac valve replacement surgery, mitral valve repair, mitral annulaplasty ring implantation, mitral leaflet “edge-to-edge” valve repair, ventricular remodeling, management of atrial appendage, septal defect repair, graft implantation, vascular anastomosis, fecal incontinence surgery, and hemorrhoid surgery. In an embodiment particularly useful for GI suturing, the device is inserted into the GI tract. In this embodiment, the tube has a diameter that can range, for example, from about 5 mm to about 22 mm for oral insertion or about 5 mm to about 33 mm for anal insertion.

Although the present invention has been described with reference to specific details of certain embodiments thereof, it is not intended that such details should be regarded as limitations upon the scope of the invention except as and to the extend that they are included in the accompanying claims. For example, although a particular feature of the invention is included in the description of one embodiment, that feature is not necessarily a limitation on the scope of the invention. Conversely, a particular feature described in one embodiment can be incorporated into any of the disclosed embodiments.

What is claimed is:

1. A suturing device for suturing within a subject, comprising:
an enclosure defining at least one suction port, wherein the suction port receives tissue within the enclosure to be sutured;
at least one cannula arranged at least partially within the enclosure, at least one needle arranged within the at least one cannula, wherein the needles are adapted to be pushed through the cannula and directed through the tissue within the enclosure to provide a suture to the tissue.

2. The suturing device of claim 1, wherein the enclosure is a tube.

3. The suturing device of claim 2, wherein the tube is flexible.

4. The suturing device of claim 2, wherein the tube is rigid.

5. The suturing device of claim 2, wherein the tube has a diameter and flexibility for insertion into an opening, and wherein the opening is at least one of a natural body orifice, a surgical incision, and an existing stoma.

6. The suturing device of claim 1, wherein the at least one cannula is a tubular passageway that allows the at least one needle to move in a forward and backward direction.

7. The suturing device of claim 2, wherein the at least one cannula directs the at least one needle to the tissue to be sutured within the enclosure.

8. The suturing device of claim 2, wherein the at least one cannula substantially prevents lateral movement of the at least one needle.

9. The suturing device of claim 2, wherein the at least one cannula is formed as part of the tube.

10. The suturing device of claim 2, wherein the at least one cannula is removable.

11. The suturing device of claim 1, wherein the at least one cannula is made of extruded plastic tubing reinforced with braided stainless wire.

12. The suturing device of claim 1, wherein the at least one cannula comprises at least one delivery cannula that extends from a portion of the suturing device that remains outside of the subject during suturing to approximately the at least one suction port.

13. The suturing device of claim 12, wherein the at least one cannula further comprises at least one receiving cannula that extends from the at least one suction port to the portion of the suturing device that remains outside of the subject during suturing.

14. The suturing device of claim 13, wherein the at least one delivery cannula and the at least one receiving cannula cooperate such that the at least one needle is passed from the at least one delivery cannula cannula, across the at least one suction port and through the tissue to be sutured, and into the at least one receiving cannula.

15. The suturing device of claim 12, wherein the at least one delivery cannula includes a bend at the distal end of the device.

16. The suturing device of claim 2, wherein the tube has a circular cross section.

17. The suturing device of claim 2, wherein the tube has a non-circular cross section.

18. The suturing device of claim 1, further comprising an endoscope for viewing the suturing.

19. The suturing device of claim 5, wherein the tube has a rigidity sufficient to enable manipulation at the opening.

20. The suturing device of claim 2, wherein the tube has an expandable diameter.

21. The suturing device of 20, wherein the at least one cannula imparts a force upon to expand the diameter of the tube.

22. The suturing device of claim 20, further comprising an inflatable balloon to control the expansion of the diameter.

23. The suturing device of claim 20, further comprising a flexible membrane spaced around the circumference of the tube above and below the at least one suction port.

24. The suturing device of claim 2, wherein the tube has a larger diameter at a distal end and narrow diameter above the at least one suction port.

25. The suturing device of claim 1, wherein the enclosure is capsule or sphere shaped.

26. The suturing device of claim 1, wherein the enclosure is pivotable.

27. The suturing device of claim 1, wherein the at least one suction port is adapted to be in fluid connection with a vacuum source such that, when a vacuum is applied, tissue is drawn into the at least one suction port and at least partially into the enclosure.

28. The suturing device of claim 27, further comprising a vacuum hose for connecting the enclosure to the vacuum source.

29. The suturing device of claim 25, further comprising an endoscope having an instrument channel, wherein the at least one cannula are arranged inside the instrument channel of the endoscope.

30. The suturing device of claim 1, wherein the at least one suction port completely circumscribes the tube.

31. The suturing device of claim 1, wherein the at least one suction port partially circumscribes the tube.

32. The suturing device of claim 1, wherein the at least one needle comprises a material with shape memory.

33. The suturing device of claim 1, wherein the at least one needle consists of a material with shape memory.

34. The suturing device of claim 1, wherein the at least one needle comprises a nickel and titanium alloy.

35. The suturing device of claim 1, wherein the at least one needle comprises one needle attached to a suture.

36. The suturing device of claim 1, wherein the at least one needle comprises two needles attached together with a suture.

37. The suturing device of claim 1, wherein the at least one cannula includes a single cannula.

38. The suturing device of claim 1, wherein the at least one cannula includes at least two cannula.

39. The suturing device of claim 1, wherein the at least one needle includes one needle arranged within the cannula.

40. The suturing device of claim 1, wherein the at least one needle includes two needles arranged within the cannula.

41. The suturing device of claim 37, wherein the at least one needle includes a needle arranged in each of the two cannulas.

42. The suturing device of claim 1, wherein the at least one cannula includes two cannulas and the at least one needle comprises two needles attached together with a suture, and wherein the two needles are backloaded into the two cannulas leaving the suture bridged between the two cannulas prior to suturing.

43. The suturing device of claim 1, wherein the at least one cannula has at least one flared opening.

44. The suturing device of claim 1, further comprising a vacuum source.

45. The suturing device of claim 1, wherein the device is sized to suture within a vessel.

46. The suturing device of claim 1, wherein the device is sized to suture within an organ or body cavity.

47. The suturing device of claim 1, wherein the needles are adapted to be manipulated from outside the subject.

48. The suturing device of claim 1, wherein the at least one suction port includes two suction ports, and wherein the device further comprises a removable partition between the suction ports.

49. The suturing device of claim 48, wherein the removable partition has a width of about 1-10 mm.

50. The suturing device of claim 1, wherein the suturing device is adapted for forming a running suture line for connecting tissue.

51. The suturing device of claim 1, wherein the suturing device is adapted for forming at least one of simple interrupted suture bites and interrupted mattress suture bites for connecting tissue.

52. The suturing device of claim 1, wherein the suturing device is adapted for forming a running suture line plicating tissue.

53. The suturing device of claim 1, wherein the suturing device is adapted for forming simple interrupted suture bites and interrupted mattress suture bites for plicating tissue.

54. The suturing device of claim 1, wherein the suturing device is adapted for closing incisions or defects.

55. The suturing device of claim 1, wherein the suturing device is adapted for connecting one lumenal tissue to a second lumenal tissue or orifice.

56. The suturing device of claim 1, wherein the suturing device is adapted for connecting tissue to a prosthetic graft.

57. The suturing device of claim 1, wherein the enclosure is formed by at least two pieces that can be separated to withdraw the suturing device from the suturing site.

58. The suturing device of claim 1, further comprising a sleeve arranged on the exterior of the enclosure for selectively covering the at least one suction port.

59. The suturing device of claim 1, further comprising a sleeve arranged on the interior of the enclosure for selectively covering the at least one suction port.

60. The suturing device of claim 1, wherein the device is adapted to narrow a diameter of at least one portion of an organ and a vessel by plication.

61. The suturing device of claim 1, wherein the at least one suction port includes two suction ports on apposing sides of the enclosure.

62. The suturing device of claim 1, wherein the at least one suction port has a length of is about 3 inches to about 9 inches in length.

63. The suturing device of claim 1, wherein the at least one suction port has a length of about ¼ inches to about 3 inches in length.

64. The suturing device of claim 1, wherein the enclosure further defines a hole in a distal end to accommodate a balloon catheter.

65. The suturing device of claim 1, further comprising an inflatable balloon.

66. The suturing device of claim 1, wherein the enclosure has a diameter of about 5 mm to about 22 mm.

67. The suturing device of claim 1, wherein the enclosure has a diameter of about 3 mm to about 32 mm.

68. The suturing device of claim 1, wherein the device is adapted to effectively reduce the volume of an organ.

69. The suturing device of claim 1, wherein the suturing results in a narrow sleeve gastroplasty.

70. The suturing device of claim 1, wherein the suturing results in a gastric pouch.

71. The suturing device of claim 1, wherein the suturing results in an augmented gastro-esophageal junction.

72. The suturing device of claim 1, wherein the suturing results in reducing the diameter of an outlet or lumen.

73. The suturing device of claim 1, wherein the enclosure includes a closed distal end.

74. The suturing device of claim 1, wherein the suture includes a suture anchor.

75. The suturing device of claim 1, wherein the at least one suture needle is of sufficient length to travel from outside the subject, pierce a tissue, and return back out of the subject.

76. The suturing device of claim 1, wherein the device adapted to be assembled and subsequently disassembled at least partially inside the subject.

77. The suturing device of claim 76, wherein the enclosure and the at least one cannula are connected with at least one of a wire, cable, string, and suture.

78. The suturing device of claim 1, wherein the at least one cannula includes an outer cannula and an inner cannula arranged to be slidably movable within the outer cannula.

79. The suturing device of claim 1, wherein the at least one cannula extends out of a distal end of the enclosure, loops and returns back into the enclosure.

80. The suturing device of claim 79, wherein the portion of the at least one cannula below the enclosure can be adjusted in length.

81. The suturing device of claim 1, wherein the cannulas running in parallel can be joined together in a ribbon pattern.

82. The suturing device of claim 1, wherein the at least one cannula includes at least one of a code and color for identification.

83. The suturing device of claim 1, wherein the at least one suture needle includes at least one of code and color for identification.

84. The suturing device of claim 1, wherein the enclosure is adapted to be manipulated by a robotic arm.

85. The suturing device of claim 1, wherein the at least one cannula can direct the at least one suture needle in one direction, have the at least one needle exit the at least one cannula, be turned around, and directed back into the at least one cannula in the opposite direction.

86. The suturing device of claim 1, wherein the at least one cannula can direct the at least one suture needle in one direction, have the at least one needle exit the at least one cannula, then be backed back into the at least one cannula.

87. The suturing device of claim 1, further comprising a protheses incorporated with the suture within the enclosure at the distal end of the device.

88. The suturing device of claim 1, wherein the at least one needle is flexible with a rigid distal segment.

89. The suturing device of claim 1, wherein a first rod in arranged within the at least one cannula to engage the at least one needle to push the needle across the at least one suction port.

90. The suturing device of claim 89, wherein, once the first rod has pushed the at least one needle across the suction port, the first rod is adapted to disengage from the at least one needle, and wherein the device further comprises a second rod arranged within the at least one cannula to engage the at least one needle to pull the needle completely across the suction port and then be able to push the needle back across the at least one suction opening.

91. The suturing device of claim 90, wherein the at least one needle is sharpened on each end and attached to the suture at approximately a mid-point of the at least one needle.

92. The suturing device of claim 90, wherein the at least one needle has a non-circular cross section.

93. The suturing device of claim 90, wherein the at least one cannula is arranged such that the at least one needle is directed horizontally across the at least one suction port.

94. The suturing device of claim 93, wherein the at least one needle is curved.

95. The suturing device of claim 93, wherein the at least one set of cannulas are aligned in a spiral fashion such that the at least one curved needle can be shuttled across a suction port.

96. The suturing device of claim 89, wherein the at least one set of cannulas are aligned in a vertical fashion such that the at least one straight needle can be shuttled across a suction port.

97. The suturing device of claim 90, wherein the at least on needle has a barb on each of its sharpened points.

98. The suturing device of claim 1, further comprising a needle catch movable forward and backward within a cannula using at least one of hydraulic pressure and pneumatic pressure.

99. The suturing device of claim 1, wherein the at least one cannula is a tubular passageway that allows a pushrod to move in a forward and backward direction.

100. The suturing device of claim 1, wherein the at least one pushrod is of sufficient length to travel from outside the subject to the suction opening.
